(19) United States
(12) Patent Application Publication
(10) Pub. No.: US 2021/0246488 A1
(43) Pub. Date: Aug. 12, 2021

(54) METHOD AND COMPOSITIONS FOR EVALUATING EMULSION UNIFORMITY

(71) Applicant: Mission Bio, Inc., South San Francisco, CA (US)

(72) Inventors: Sebastian Treusch, San Francisco, CA (US); Adam R. Sciambi, San Francisco, CA (US); Maurizio Pellegrino, San Francisco, CA (US); Dennis Jay Eastburn, Burlingame, CA (US)

(73) Assignee: Mission Bio, Inc., South San Francisco, CA (US)

(21) Appl. No.: 16/639,422

(22) PCT Filed: Aug. 14, 2018
(86) PCT No.: PCT/US2018/046762

§ 371 Date: Feb. 14, 2020

Related U.S. Application Data
(60) Provisional application No. 62/545,292, filed on Aug. 14, 2017.

(51) Int. Cl. C12Q 1/6806; C12Q 1/6853

(52) U.S. Cl. CPC C12Q 1/6806; C12Q 1/6853

(57) ABSTRACT

The disclosure relates to methods and compositions for evaluating emulsion uniformity. In an exemplary embodiment, the disclosure provides a method for evaluating a quality characteristic of a droplet. The method includes the steps of: encapsulating a plurality of polynucleotides in a droplet, wherein the plurality of polynucleotides comprises at least one species of oligonucleotide; tagging the polynucleotides with a label that identifies the polynucleotides as arising from the droplet; counting a number of species of oligonucleotide tagged with the label; and determining a quality characteristic of the droplet based on the number of species of oligonucleotide tagged with the label. The oligonucleotide may include a first nucleic acid segment and a second nucleic acid segment, wherein the first nucleic acid segment comprises a plurality of random nucleotides; and the second nucleic acid segment comprises a conserved region common to the plurality of oligonucleotides.

### CROSS REFERENCE

This application claims the benefit of U.S. Provisional Application No. 62/545,292, filed Aug. 14, 2017, which application is incorporated herein by reference.

### BACKGROUND

Microfluidic control over emulsion generation enables the creation of highly monodisperse droplet populations. This results in precise partitioning of a biological sample, which can include single cells or other material loaded into droplets at Poisson statistics. These techniques allow different single-droplet or single-cell analytical techniques to be performed on the materials at high-throughput. These techniques can be improved when the emulsions are stable and maintain proper partitioning.

However, maintaining emulsion stability while conducting complex biochemical reactions within droplets can be challenging. Changes in temperature, environmental dust, physically handling or manipulating samples, and ambient static charges are examples of things that can lead to droplet merger during a workflow. Droplet merger can result in the contents of two or more droplets combining. This can result in the mixing of information from multiple droplets and the loss of single-droplet resolution, including the loss of single cell resolution.

Thus, for many applications, it can be advantageous to detect events that can affect single-droplet resolution in the data, including variations in droplet size, reagent addition, and unintended droplet merging.

### SUMMARY

The compositions and methods described herein allow for the assessment of quality characteristics of droplets, including characteristics related to droplet formation, uniformity, and manipulation during a workflow. For example, the compositions and methods can be used to determine the volume of a fluid used to form a droplet or detect events like droplet merger or reagent addition to droplets during a workflow.

The methods generally include the use of synthetic oligonucleotide species containing a highly-variable segment. The oligonucleotides can be added to the fluids used to form droplets at a Poisson distribution consistent with a volume of the fluid used to make or added to a droplet. The oligonucleotides can also include regions that are held in common between oligonucleotides within the same droplet, such as a droplet-specific label. Such labels, which can include barcodes, can be informative of the droplet from which the oligonucleotide arose. In some cases, the oligonucleotides can also include regions that are held in common between oligonucleotides arising from the same fluid used in a workflow. As such, these regions can be informative of the fluid from which the oligonucleotides arose.

The methods generally include sequencing the resulting oligos. Quantifying a number of oligonucleotide species containing the same droplet-specific label can be informative of a volume characteristic of the droplet. The volume of a droplet can be indicative of inconsistency in droplet formation size or droplet merger. Detecting individual oligonucleotide species that are labeled with more than one droplet-specific label can be informative of a droplet containing two or more droplet-specific labels. This can result from droplet merger or from droplets being formed with two or more labels.

Detecting such events can help with downstream analysis of sequences generated from droplets. For example, detecting droplet mergers can be informative of a droplet containing more than one cell, which can reduce or eliminate the single cell resolution of the nucleic acids arising from that droplet. The analysis can then correct or eliminate sequencing data arising from that droplet from further analysis. As a result, the methods can be used to assess the quality of various characteristics of droplets used in a workflow, which can increase the accuracy of or confidence in the resulting data.

Provided herein are methods of evaluating a quality characteristic of a droplet comprising: encapsulating a plurality of polynucleotides in a droplet, wherein the plurality of polynucleotides comprises at least one species of oligonucleotide; tagging the polynucleotides with a label that identifies the polynucleotides as arising from the droplet; counting a number of species of oligonucleotide tagged with the label; and determining a quality characteristic of the droplet based on the number of species of oligonucleotide tagged with the label.

In some embodiments, an oligonucleotide comprises: a first nucleic acid segment and a second nucleic acid segment, wherein the first nucleic acid segment comprises a plurality of random nucleotides; and the second nucleic acid segment comprises a conserved region common to the plurality of oligonucleotides.

In some embodiments, the method further comprises forming the droplet in a microfluidic device. In some embodiments, forming the droplet comprises isolating a portion of a first fluid, wherein the first fluid comprises a known concentration of oligonucleotide species. In some embodiments, the first fluid comprises a plurality of the species of oligonucleotide dispersed throughout the first fluid, and wherein isolating the portion of the first fluid comprises isolating a number of oligonucleotide species in the droplet according to a Poisson distribution that is proportional to the volume of the droplet. In some embodiments, the droplet comprises an aqueous phase fluid dispersed in an immiscible phase carrier fluid.

In some embodiments, the droplet comprises a known concentration of oligonucleotides. In some embodiments, the plurality of polynucleotides comprises a number of polynucleotides encapsulated according to a Poisson distribution dependent on a volume of the droplet. In some embodiments, the number of species of oligonucleotide tagged with the label is informative of a volume of the droplet. In some embodiments, the volume of the droplet comprises a volume of the immiscible phase carrier fluid.

In some embodiments, the plurality of polynucleotides further comprises polynucleotides obtained from a sample. In some embodiments, the sample comprises a cell. In some embodiments, the sample comprises no more than one cell. In some embodiments, encapsulating the plurality of polynucleotides in the droplet comprises encapsulating a cell comprising polynucleotides in the droplet. In some embodiments, the method further comprises lysing the cell in the droplet.

In some embodiments, the label comprises a barcode. In some embodiments, tagging the polynucleotides with a label comprises subjecting the droplet to conditions sufficient for enzymatic incorporation of the label into the plurality of polynucleotides. In some embodiments, enzymatic incorporation of the label into the plurality of polynucleotides comprises ligating the label to the polynucleotides. In some embodiments, tagging the polynucleotides with a label comprises subjecting the droplet to conditions sufficient for enzymatic incorporation of the label into amplification products of the plurality of polynucleotides. In some embodiments, enzymatic incorporation of the label into amplification products of the plurality of polynucleotides comprises ligating the label to the amplification products. In some embodiments, enzymatic incorporation of the label into amplification products of the plurality of polynucleotides comprises amplifying the plurality of polynucleotides by PCR using barcoded primers.

In some embodiments, counting a number of species of oligonucleotide tagged with the label comprises sequencing the polynucleotides tagged with the label. In some embodiments, the quality control characteristic is a volume of the droplet. In some embodiments, the quality control characteristic is a merger of two droplets. In some embodiments, data arising from the droplet is adjusted based on the quality characteristic. In some embodiments, the quality characteristic comprises a volume of the droplet. In some embodiments, the quality characteristic comprises a droplet merger. In some embodiments, data arising from the droplet is excluded from further analysis based on the quality characteristic. In some embodiments, the quality characteristic comprises a volume of the droplet. In some embodiments, the quality characteristic comprises a droplet merger.

Also provided herein are methods of evaluating a quality characteristic of a droplet comprising: sequencing a plurality of polynucleotides obtained from the droplet, wherein the plurality of polynucleotides comprises at least one oligonucleotide species comprising a first nucleic acid segment and a second nucleic acid segment, wherein the first nucleic acid segment comprises a plurality of random nucleotides; and the second nucleic acid segment comprises a conserved region comprising a label, detecting sequences of oligonucleotide species comprising labels; and determining a quality characteristic of the droplet based on the sequences of the oligonucleotide species detected.

In some embodiments, the plurality of polynucleotides comprises a first oligonucleotide species comprising a first conserved region and second oligonucleotide species comprising a second conserved region, wherein the detecting of sequences encoding the first oligonucleotide species and second oligonucleotide species is informative of a droplet merger. In some embodiments, the first conserved region comprises a first label and the second conserved region comprises a second label. In some embodiments, the first label comprises a first barcode and the second label comprises a second barcode. In some embodiments, the first oligonucleotide species comprises a first label indicative of a first group of droplets and a droplet-specific label and the second oligonucleotide species comprises a second label indicative of a second group of droplets and the droplet specific label. In some embodiments, the quality characteristic is a merger between a droplet from the first group of droplets and a droplet from the second group of droplets. In some embodiments, the plurality of polynucleotides further comprises polynucleotides obtained from a sample. In some embodiments, the sample comprises a cell.

In some embodiments, the droplet is a merged droplet, and wherein the method further comprises an intentional merger between a first droplet comprising the first oligonucleotide species and a second droplet comprising the second oligonucleotide species to form the merged droplet. In some embodiments, the first droplet comprises polynucleotides obtained from a sample and the second droplet comprises reagents. In some embodiments, the first droplet comprises polynucleotides obtained from a first sample and the second droplet comprises polynucleotides obtained from a second sample. In some embodiments, the droplet is a merged droplet resulting from an unintentional merger between a first droplet comprising the first oligonucleotide species and a second droplet comprising the second oligonucleotide species to form the merged droplet. In some embodiments, the plurality of polynucleotides comprises plurality of polynucleotides encoding a first oligonucleotide species, wherein at least one member of the plurality of nucleotides encoding the first nucleotide species is labeled with a first label and at least one member of the plurality of nucleotides encoding the first nucleotide species is labeled with a second label. In some embodiments, detecting at least one member of the plurality of nucleotides encoding the first nucleotide species labeled with the first label and at least one member of the plurality of nucleotides encoding the first nucleotide species labeled with the second label is informative of droplet merger.

In some embodiments, the droplet is a merged droplet and wherein the first label is informative of a first droplet and the second label is information of a second droplet, and wherein the first droplet and the second droplet merged to form the merged droplet. In some embodiments, the droplet merger is unintentional. In some embodiments, at least one of the first label and second label comprises a barcode. In some embodiments, the first droplet comprises polynucleotides obtained from a first sample and the second droplet comprises polynucleotides obtained from a second sample. In some embodiments, data arising from the droplet is adjusted based on the quality characteristic. In some embodiments, the quality characteristic comprises a volume of the droplet. In some embodiments, the quality characteristic comprises a droplet merger. In some embodiments, data arising from the droplet is excluded from further analysis based on the quality characteristic. In some embodiments, the quality characteristic comprises a volume of the droplet. In some embodiments, the quality characteristic comprises a droplet merger.

Also provided herein are compositions. In some embodiments, the composition comprises a droplet comprising at least one oligonucleotide species comprising a first nucleic acid segment and a second nucleic acid segment, wherein the first nucleic acid segment comprises a non-conserved region; and the second nucleic acid segment comprises a conserved region, and wherein a number of oligonucleotide species present in the droplet is indicative of a quality control characteristic of the droplet.

In some embodiments, the non-conserved region comprises a plurality of random nucleotides. In some embodiments, the conserved region comprises a primer binding site. In some embodiments, the droplet further comprises a plurality of first primers, wherein each primer comprises a region that is complementary to the primer binding site. In some embodiments, each first primer further comprises a barcode that is common to the plurality of first primers. In some embodiments, the oligonucleotide species further comprises a barcode common to the oligonucleotide species present in the droplet.

In some embodiments, the droplet comprises an aqueous droplet. In some embodiments, the aqueous droplet is surrounded by an oil. In some embodiments, the droplet further comprises a DNA polymerase. In some embodiments, the droplet further comprises a reverse transcriptase.

In some embodiments, the quality control characteristic comprises detecting a merger of two droplets. In some embodiments, the quality control characteristic comprises a volume of the droplet.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts expected results obtained from various events during droplet formation. The diagram includes the events, the readout obtained from sequencing the drop, and the inferred origin of the drop. By barcoding drops containing sets of randomized oligos (RO), each drop's origin can be traced via downstream sequencing. A drop can be found to be (a) intact throughout a workflow, (b) the result of three drops merging, (c) the precise pairing of two drop types, or (d) a more complex combination.

FIG. 2A-2B depicts the results of a method of determining the likelihood that a drop formed by various events. Using Poisson statistics to identify the size and origin of a drop containing (a) 7 ROs given a mean occupancy of 3 and (b) 28 ROs given a mean occupancy of 10. In (a), the drop in question is likely a merger, whereas in (b), it is very likely due to a merger. The increased resolving power of higher occupancy in (b) means more confidence can be placed in the results than those determined in (a).

FIG. 3 depicts theoretical results calculated for a variety of different numbers of random oligos loaded per unit drop. (a) Distributions of possible occupancies of drops of some size (peaks labeled 1×, 2×, 3×, 5×, and 10×) and the ranges that indicate what the size of that drop is called for different average RO loading (numbers at the top of each chart). (b) The likelihood that a drop of some actual size is detected as another size.

### DETAILED DESCRIPTION

Disclosed herein are methods, compositions, and systems wherein barcoded droplets are loaded with oligonucleotides to track the droplets' characteristics. These include sizes and eventual states as intact partitions, origins, contents, intentionally combined mergers, or unintentional mergers, by characterizing the oligonucleotide species and labels using downstream sequencing.

Droplet microfluidics makes use of the partitioning of samples and reagents into microscale compartments in which millions of reactions can occur in parallel. To keep this parallelization from being muddled, workflows benefit from partitions that remain fully intact during the entire reaction. Some droplet microfluidic workflows benefit from the formation of monodisperse droplets with consistent sizes. The ability to assess the consistency of droplet sizes can increase the confidence one has that each droplet was created, handled, and processed similarly. The detection of inconsistent droplet formation can allow for troubleshooting and the exclusion or normalization of data generated from improperly formed droplets during downstream analysis.

Some droplet workflows include the addition of reagents to droplets after they are initially formed. For example, in some workflows, cells can be combined with lysis reagents during droplet formation and incubated under conditions that allow the cells to lyse inside the droplet, thereby releasing molecules like nucleic acids into the droplet. Additional reagents, such as reagents useful for reverse transcribing RNA, synthesizing or amplifying nucleic acids, and/or detecting nucleic acids are sometimes added to droplets after the cells have been lysed. Thus, it can be advantageous to detect and assess the consistency with which these subsequent reagents are added to each droplet.

Single-cell analysis is an example where the unintentional merger of two or more single-cell-containing droplets during processing can result in an inability to identify the original cell populations. The methods described herein can include detecting intact drops in an emulsion or inferring the constituent droplets that merged. The methods and compositions described herein can be used to assess unintended droplet merging events in single-droplet or single-cell sequencing techniques that include molecular barcodes. Sequences obtained from a droplet determined to likely be the result of an unintended merger can be excluded from further analysis or appropriate corrections made.

Alternatively, some workflows require droplets from disparate populations to be precisely paired or combined in larger numbers. For example, some workflows include merging individual drops containing prepared barcode libraries with drops containing individual cells. Improper pairing can lead, for instance, to multiple cells tied to a single barcode or multiple barcodes to a single cell.

It can also be beneficial to distinguish droplets that vary as to their nucleic acid content so as to identify the density or amount of nucleic acids relative to the volume of the droplet.

With these examples in mind, it can be advantageous when analyzing a final population to be able to distinguish ideal droplets from those that are unintentionally merged, merged incorrectly, contain incorrect or suboptimal components, or were properly formed but are simply the wrong size.

This disclosure presents methods that generally use unique or rare oligonucleotides (ROs) to identify microfluidic events that can compromise data analysis. At least a segment or portion of the RO can contain a highly randomized sequence. The RO often also contains at least one segment or portion that is constant or consistent across a plurality of other oligonucleotides. Such constant regions can comprise primer binding sites or sites compatible with attaching a barcode.

The ROs are generally loaded into droplets with a Poisson distribution. As a result, each microfluidically-generated mono disperse droplet usually obtains a number of ROs that depends on the concentration of oligo used and the droplet volume. Generally, because the same fluid with a consistent concentration of ROs is used to create or add reagents to each droplet within a population of droplets, the number of ROs added to each droplet can be indicative of the amount of volume used in that particular step.

The contents of each droplet, including the samples and the ROs, can be labeled using droplet-specific barcodes. In some cases, the barcode is unique to a particular droplet within a larger population of droplets. In other cases, the barcode need not be unique but can still be used to identify the droplet of origin. Thus, the barcode can form an association between the droplet, the ROs, and the target sample nucleic acids contained in the droplet. The barcoded ROs can then be identified and counted in post-processed droplets, such as via downstream sequencing, to discern the history of the drop. When the number of RO sequences associated with a single droplet barcode is higher than the expected number, this can indicate a likelihood that the droplet volume was larger than the intended monodisperse partition size. The larger volume may be due to droplet merger, problems with droplet formation uniformity, or problems with fluid addition uniformity, among others. Various embodiments are described in more detail below that can distinguish between these events in some cases.

The methods often employ the use of fluids containing ROs at a fixed starting concentration. The fluids can be dispensed from a master mix or a single reservoir containing a stock solution. By adding ROs from this starting concentration to drops, the number of ROs matched to a particular barcode can roughly correlate with the volume of fluid added to each drop. Drops often begin the methods monodisperse and roughly uniform in size. As such, the volume of the final drop can indicate the number of drops that merged to make that final drop. These methods can also be used to determine the size of drops of various volumes, such as those created in polydisperse, shaken emulsions, or in emulsions in which mergers were not expected to occur.

Stable droplets keep their contents properly partitioned from other droplets. The methods described herein can be used to detect improper droplet merger. For example, the merger can include two drops of average size, each drop containing an average number of ROs. In such cases, the total number of ROs contained in the merged drop will be twice the number expected.

A single stable droplet containing a single set of unique ROs and single set of droplet-specific barcodes should yield a population of ROs, each labeled with the droplet-specific barcode. Thus, the detection of an RO labeled with more than one barcode can be informative of a variety of possible events. In one example, the detection of an RO species associated with barcodes associated with more than one drop can indicate droplet merger. Such a merger can involve the merger of two drops, each drop containing its own droplet specific barcode. The merged drop that results, therefore, may contain two different sets of barcodes instead of one. If the RO is amplified and copies are found associated with more than one RO, this could be due to droplet merger. Alternatively, such as result may also be informative of barcoding fidelity.

As another exemplary use, ROs can be used to detect the merging of two or more sets of different drop populations. In some of the methods described herein, individual droplets or subpopulations of droplets can be loaded with their own distinguishable set of ROs. These ROs can contain a combination of a first segment containing non-conserved (for example, random) nucleic acids and a second segment containing conserved nucleic acids held in common between the ROs of the set. The conserved nucleic acids can identify the ROs as belonging to the set. In such methods, detecting members of different subpopulations of ROs in the same final droplet can be informative of the identity or contents of the individual droplets that merged to form the final droplet.

In addition to ROs, in some cases each drop contains one or more sets of labels, such as drop-specific sequences, that can attach to the ROs and be used to group of bin sequences for later identification.

The methods and compositions described herein offer several advantages. As an exemplary advantage, the ROs can be added to the drop reagent mix used to form the droplets. Thus, the ROs can be added to droplets without altering existing or predetermined microfluidics. As another exemplary advantage, the ROs can be barcoded, detected, and sequenced using conventional barcoding protocols. As a further exemplary advantage, mergers between intentionally combined droplets or droplet subpopulations can have the composition of the resulting droplet inferred.

### 1. Oligonucleotides Containing Identifying Sequences

Provided herein are oligonucleotides (ROs) useful in the assessment of a quality control characteristic of the isolated reaction volume, such as a droplet. The ROs can be either single or double stranded. ROs can comprise DNA, RNA, or mixed DNA/RNA molecules. ROs can also comprise standard or modified nucleotides.

In general, the ROs comprise a first segment containing a sequence that allows for each individual RO species to be identified and distinguished from other ROs in a population contained within a single droplet, a plurality of droplets, a sample, an experiment, or a sequencing run. The first segment is often unique within a population of ROs, although it need not be completely unique.

The first segment can be a region comprising randomized nucleotides. The first segment can be synthesized by randomly incorporating a mixture of nucleotides, including A, C, G, T, non-standard nucleotides, and analogs thereof. The first segment can also be synthesized with a predefined or known sequence.

The length of the first segment can vary depending on, for example, the number of RO species desired, the amount of diversity desired between the RO species, and the sequencing methods to be used. In some instances, the suitable diversity of the first segment is on the order of ≥4¹⁰, representing a 10 base randomized oligomer. In some cases, the RO comprises a first segment with 2, 3, 4, 5, 6, 7, 8, 9, 10, 11, 12, 13, 14, 15, or more than 15 bases. In some cases, the RO comprises a first segment with a number of bases corresponding to any integer between 3 and 150. In some cases, the RO comprises a first segment with a number of bases corresponding to any integer between 3 and 4000.

The RO generally includes a second segment containing a region common to a plurality of the ROs. The region can be common to an entire population of ROs, a sub-population of ROs' or can be specific to a sample, fluid, or step used in one of the methods described herein. The second segment can comprise an adaptor, a primer binding site, a restriction site, and/or a site partially complementary to a sequence on a barcode.

The primer binding site can include a sequence complementary to a primer used for sequencing. Alternatively or in addition, the primer binding site can include a sequence complementary to a primer used to amplify the RO. As will be described in more detail, some of the primers that bind to the primer binding site can include a barcode, such as a drop-specific or sample-specific barcode. Thus, in some cases, the primer binding site can be used to tag the RO with a barcode during an amplification step.

The second segment can also include an oligo tag common to the plurality of ROs. Oligo tags can be barcodes, as described above. Oligo tags can serve to identify a fluid used during the formation or processing of a droplet. For example, a first class of ROs with a first common oligo tag can be added to an aqueous fluid carrying cells from a sample. A second class of ROs with a second common oligo tag can be added to a lysis solution. A third class of ROs with a third common oligo tag can be added to a solution containing PCR reagents. As will be described in more detail below, the different oligo tags allow ROs to be classified and identified as originating from individual solutions. Thus, when the solution containing cells is combined with lysis solution and formed into droplets, the oligo tags can help identify ROs from the cell solution separately from ROs from the lysis buffer. After lysis completes, the droplet can be merged with a second droplet containing PCR reagents. The ROs in the PCR reagent drop contain a third oligo tag common to the ROs in the PCR reagent drop. The different oligo tags in each solution allow for characteristics of each individual solution that forms the final droplet to be separately assessed in the final droplet.

The RO can comprise a third segment containing a region common to a plurality of the ROs. The region can be common to an entire population or ROs, a sub-population of ROs' or can be specific to a sample, fluid, or step used in one of the methods described herein. The third segment can comprise a primer binding site, a restriction site, a barcode, and/or a site partially complementary to a sequence on a barcode. In some cases, the second segment is located 5′ to the first segment and the third segment is located 3′ to the first segment. In some cases, the second segment is located 3′ to the first segment and the third segment is located 5′ to the first segment.

Alternately or in combination, different versions of the synthetic oligonucleotides with different constant sequences rather than only one version are contemplated, as discussed above. Having different constant regions that facilitate the association with the barcoding tags increases robustness by counteracting DNA sequence biases that influence sequence association. That is, in some cases multiple gBlocks are amplified using different primers.

The synthetic molecule is in various embodiments composed of one or more than one randomized region (such as 2, 3, 4, 5, 6, or more than 6 randomized regions). The non-variable regions in between the more than one randomized regions can be used to estimate the amount of error introduced during the manipulation of the molecule itself. For example, if the non-variable regions comprise known sequences, sequences of the non-variable regions that contain errors can be used to infer the error rate of the assay.

The constant regions of the synthetic molecule are frequently used to identify the synthetic oligo within the next-generation sequencing results and associate it with the appropriate barcoding tag. Errors in the synthetic random sequences are often corrected using algorithms such as Hemming distance or Levenshtein distance based on estimated error rates. Other approaches are known and consistent with the disclosure herein. Sequences that are found to come from the same synthetic oligo and associated with the same barcode can, therefore, be grouped together.

### 2. Methods of Evaluating Emulsions

Disclosed herein are methods of evaluating droplet quality characteristics. Generally, each droplet includes an aqueous phase fluid in an immiscible phase carrier fluid. Examples of such characteristics can include properties related to the formation of droplets, properties related to the workflow of a particular method or manipulation, or properties of a final droplet after a method is complete. In particular, the characteristics can relate to the volume of a droplet, the volume of fluid added to a droplet, the contents of a droplet, intentionally combined droplet or fluid mergers, and unintentionally combined droplet or fluid mergers. In some cases, the methods can identify characteristics, such as identity or volume, of the droplets or fluids that are combined or merged to form a droplet.

Droplets can be isolated reaction volumes and often include an aqueous fluid surrounded by an oil or an organic phase. In some cases, the aqueous droplet is emulsified in an oil, liposome, lipid bilayers, and micelles. Droplets are often formed in a microfluidic device.

The composition and nature of the discrete entities, e.g., droplets, e.g. microdroplets, prepared and/or utilized in connection with the disclosed methods may vary. For example, in some embodiments, a droplet may include one cell and not more than one cell. In other embodiments, a droplet may include a plurality of cells, i.e., two or more cells. In some aspects, droplets according to the present disclosure may include a nucleic acid or a plurality of nucleic acids. In some embodiments, as discussed herein, droplets may include one or more solid and/or gel materials, such as one or more polymers.

In some embodiments, a surfactant may be used to stabilize the droplets. Accordingly, a droplet may involve a surfactant stabilized emulsion. Any convenient surfactant that allows for the desired reactions to be performed in the droplet may be used. In other aspects, a droplet is not stabilized by surfactants or particles.

The surfactant used depends on a number of factors such as the oil and aqueous phases (or other suitable immiscible phases, e.g., any suitable hydrophobic and hydrophilic phases) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures.

Other surfactants can also be envisioned, including ionic surfactants. Other additives can also be included in the oil to stabilize the droplets including polymers that increase droplet stability at temperatures above 35° C.

The droplets described herein may be prepared as emulsions, e.g., as an aqueous phase fluid dispersed in an immiscible phase carrier fluid (e.g., a fluorocarbon oil or a hydrocarbon oil) or vice versa.

Emulsions may be generated using microfluidic devices. Microfluidic devices can form emulsions made up of droplets that are extremely uniform in size. The droplet generation process may be accomplished by pumping two immiscible fluids, such as oil and water, into a junction. The junction shape, fluid properties (viscosity, interfacial tension, etc.), and flow rates influence the properties of the droplets generated but, for a relatively wide range of properties, droplets of controlled, uniform size can be generated using methods like T-junctions and flow focusing. To vary droplet size, the flow rates of the immiscible liquids may be varied since, for T-junction and flow focus methodologies over a certain range of properties, droplet size depends on total flow rate and the ratio of the two fluid flow rates.

To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating droplet of the desired size and rate. The nature of the microfluidic channel (or a coating thereon), e.g., hydrophilic or hydrophobic, may be selected so as to be compatible with the type of emulsion being utilized at a particular point in a microfluidic work flow.

In some embodiments, droplets are generated using a droplet maker as described in PCT Publication No. WO 2014/028378, the disclosure of which is incorporated by reference herein in its entirety and for all purposes.

The methods generally include the use of the ROs described above. Droplets are generally loaded with ROs to track the droplets' sizes and eventual states as intact partitions, intentionally combined mergers, or unintentional mergers, by counting the number of oligonucleotide species using downstream sequencing. The methods often include flowing an amount of a fluid comprising a concentration of ROs to form a droplet, tagging the ROs in a droplet-specific manner, sequencing the ROs, and counting the number of ROs containing the droplet specific barcode. The methods also include adding an amount of a fluid comprising a concentration of ROs to a droplet that has already been formed. ROs, like other reagents, are generally loaded into droplets following Poisson statistics when there is a uniform or generally consistent concentration of non-interacting ROs being partitioned into droplets. As a result, the number of ROs present in a droplet can be informative of the volume of fluid used to form the droplet.

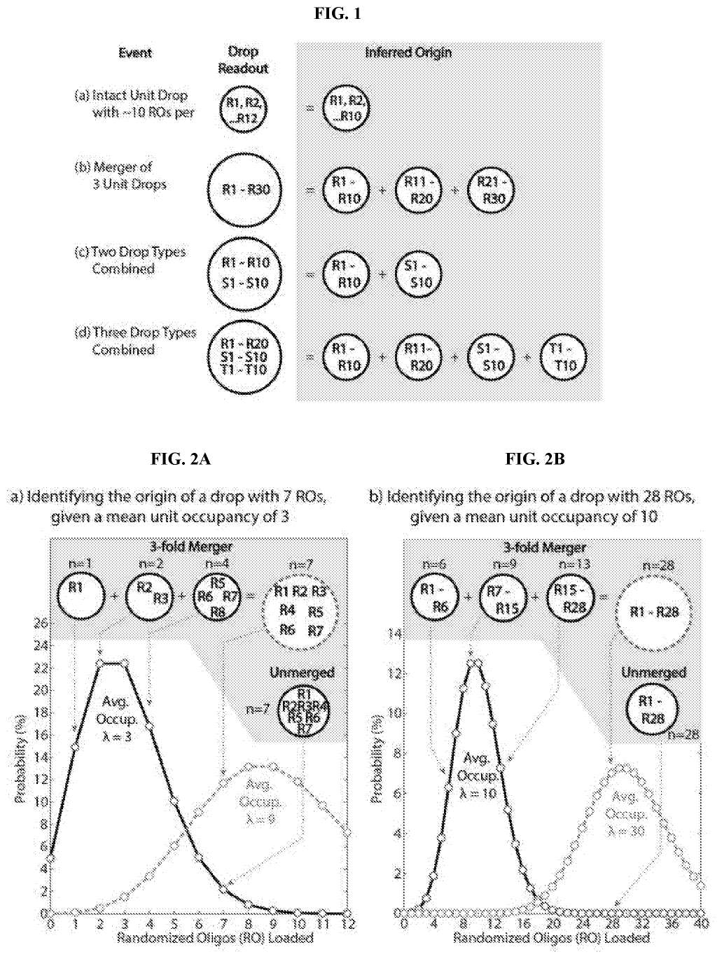

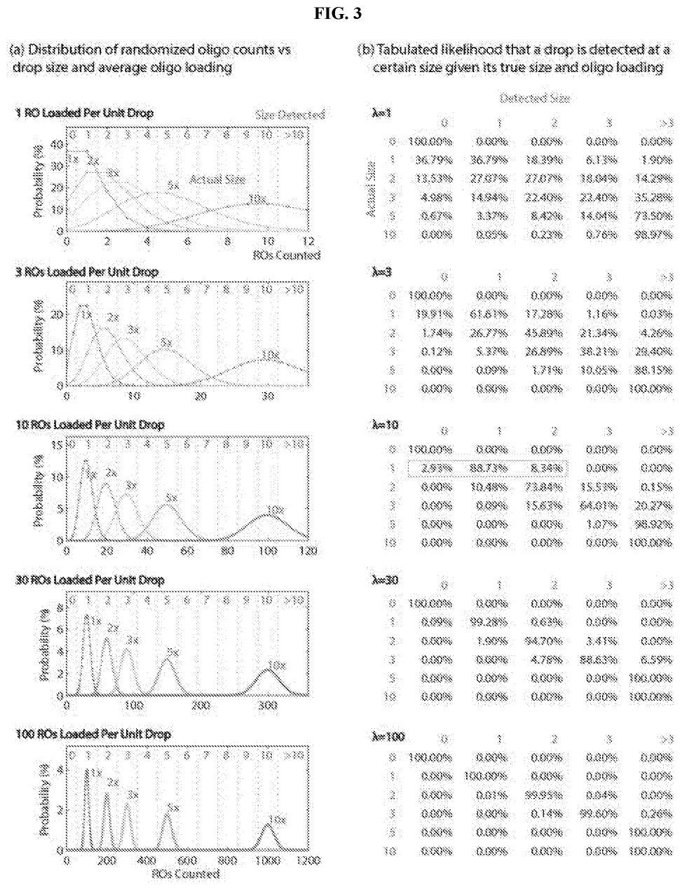

The ROs are generally added at a known concentration to the fluid for which determining a quality characteristic is desired. The fluid can be a fluid used to form the droplet, a single stream of fluid merged with at least one other stream of fluid to form a droplet, or a fluid added to a droplet after it has been formed. The concentrations are generally calibrated to yield a mean number of ROs present in unit drops. The targeted mean number of ROs present in unit drops can vary depending on the types of characteristics that are desired to be analyzed. As described in Example 12 and FIG. 2 and FIG. 3, the likelihood that the volume of a drop of a particular size is correctly determined can depend upon the average number of ROs loaded in each drop.

A. Labeling Nucleic Acids

In some embodiments of the methods described herein, the methods include labeling target nucleic acids. The methods often include droplets containing nucleic acids derived from a sample, such as DNA or RNA isolated or amplified from a cell. Such nucleic acids can be labeled using the methods described herein. The methods can also include labeling the ROs. The ROs can sometimes be synthesized with droplet or step-specific sequences.

The labeling is generally done in a droplet-specific manner. As such, target nucleic acids within a droplet are labeled with a common label while target nucleic acids in different droplets are generally labeled with different labels. Thus, sample-derived nucleic acids and ROs in the same droplet can be labeled with the same label while sample-derived nucleic acids and ROs in different droplets can be labeled with different labels. The labels need not be absolutely unique in a sample or experiment, but are generally diverse enough such that target nucleic acids in different droplets can be distinguished from each other. The label generally is or comprises a nucleic acid barcode.

(i) Labels

Also provided herein are labels used to label polynucleotides, such as the ROs or other nucleotides of interest. As used herein, the terms “label,” “barcode”, “unique molecular identifier,” “UMI,” or “molecular tag” refer to a nucleic acid sequence that allows some feature of a polynucleotide with which the barcode is associated to be identified. For example, the barcodes can be used to label ROs and target nucleic acids, including nucleic acids derived from a cell.

In the present disclosure, barcodes often label polynucleotides in a droplet-specific manner. In certain cases, a droplet comprises a plurality of oligonucleotides comprising a molecular tag or barcode. In many cases, these barcoded oligonucleotide molecules within a droplet have identical sequences. In further embodiments, the barcoded oligonucleotide molecules have identical molecular tag or barcode sequences. In other cases, the droplet includes barcoded oligonucleotides that sort into at least two populations, each population characterized by a distinct barcode sequence.

The barcodes can be a predetermined sequence or can be a random or degenerate sequence. In general, barcodes are of sufficient length and comprise sequences that are sufficiently different to allow the identification of samples based on barcodes with which they are associated. Barcodes are often at least 3, 4, 5, 6, 7, 8, 9, 10, 11, 12, 13, 14, 15, or more nucleotides in length. In some cases, barcodes are at least 10, 11, 12, 13, 14, or 15 nucleotides in length, while in other cases barcodes are shorter than 10, 9, 8, 7, 6, 5, or 4 nucleotides in length. In certain examples, barcodes are shorter than 10 nucleotides in length. In some embodiments, barcodes associated with some polynucleotides are of different length than barcodes associated with other polynucleotides.

With respect to sequence diversity, each barcode in a plurality of barcodes can sometimes differ from every other barcode in the plurality. In some cases, each barcode differs from the other barcodes by at least two nucleotide positions, such as at least 2, 3, 4, 5, 6, 7, 8, 9, 10, or more positions. In some cases, each barcode differs from every other barcode by in at least 2, 3, 4 or 5 positions.

In some cases, the barcodes are part of a larger oligonucleotide molecule. For example, the oligonucleotide molecule may include a barcode portion and a conserved portion. The conserver portion can include a primer, such as a primer that is complementary to a primer binding site on an RO or a target nucleic acid molecule.

Also provided herein are barcode aggregates (also referred to as “barcode balls), each comprising multiple copies of a barcode. Such barcode aggregates can be useful when introducing multiple copies of a barcode into a single droplet. Examples of barcode aggregates include barcoded hydrogel microspheres, including those described in Klein et al., 2015, Cell 161, 1187-1201. Also provided are libraries of barcode aggregates.

Various methods of labeling ROs are contemplated herein. In some cases, the methods include providing a plurality of barcodes to a droplet. Exemplary methods of providing barcodes are described below. The label can be attached to the ROs using a variety of methods, some examples of which are described below. For example, the methods can include amplifying the ROs using primers comprising the label. As a result, each round of amplification can amplify a single template molecule, such as an RO, to produce an amplicon comprising the RO sequence and the label.

To accomplish amplification, reagents sufficient for amplification may also be included in the droplets, such as enzymes necessary for thermal cycled amplification, including thermostable polymerases, or isothermal amplification, such as polymerases for multiple-displacement amplification. Other, less common forms of amplification may also be applied, such as amplification using DNA-dependent RNA polymerases to create multiple copies of RNA from the original DNA target which themselves can be converted back into DNA, resulting in, in essence, amplification of the target. Living organisms can also be used to amplify the target by, for example, transforming the targets into the organism which can then be allowed or induced to copy the targets with or without replication of the organisms. The degree of amplification may also be controlled by modulating the concentration of the amplification reagents to achieve a desired level of amplification. In some instances, this is useful for fine tuning of the reactions in which the amplified products are used.

Suitable amplification methods for use with the disclosed methods may include, e.g., DNA polymerase PCR, RecA-mediated recombination PCR, helicase displacement PCR, and/or strand displacement based template amplification methods, including, but not limited to Multiple Displacement Amplification (MDA), Multiple Annealing and Looping-Based Amplification Cycles (MALBEC), rolling circle amplification, nick-displacement amplification, and Loop-Mediated Isothermal Amplification (LAMP).

Accordingly, in some embodiments the present disclosure provides a method for producing compartmentalized, amplified target libraries for barcode-based sequencing, wherein the method includes (a) encapsulating a plurality of nucleic acid target molecules comprising at least one RO in a plurality of discrete entities with reagents sufficient for the enzymatic amplification of the nucleic acid target molecules; (b) subjecting the discrete entities to conditions sufficient for enzymatic amplification of the nucleic acid target molecules, providing amplification products; (c) fragmenting the amplification products; and (d) incorporating nucleic acid barcode sequences into the fragmented amplification products.

In some aspects, the methods include the detection of individual RO species that are labeled with more than one label. For example, some ROs can be contained in a droplet containing two different label species, such as two different barcode species. This may be the result of, for example, merger between two different droplets each of which contained a droplet-specific label. In such cases, it may be desirable to know if a single droplet contains more than one label. Thus, the methods may include amplifying the ROs contained within a droplet prior to the labeling step. The amplicons produced can be labeled with a label. Where multiple labels are present in a droplet, some amplicons of an RO may be labeled with one label and other amplicons may be labeled with a different label. The detection of copies of a species of RO with different labels can indicate that multiple labels were present in the droplet in which the RO was present. This can be informative of a quality characteristic of the droplet. The quality characteristic can include merger of two different droplets or loading a single droplet with two different label species.

Alternatively or in addition, the labeling technique can be repeated multiple times. For example, in the case of labeling using PCR with barcoded primers, a first primer with a first barcode sequence can anneal to a template molecule, such as an RO. The resulting amplicon will include the first barcode label. If more than one species of barcoded primer is present, a second primer with a second barcode sequence can anneal to the same template molecule, for example, during a subsequent amplification or labeling cycle. The resulting amplicon will include the second barcode label. Thus, the detection of copies of a species of RO with different labels can indicate that multiple labels were present in the droplet in which the RO was present. This can be informative of a quality characteristic of the droplet.

Alternatively or in addition, labels can be directly incorporated into the template molecules. For example, labels can be ligated directly to ROs or other target molecules. In some cases, more than one label can be ligated to an end of a target molecule. In another aspect, ROs can be amplified within a droplet and labels directly incorporated into the resulting amplicons. If more than one label species is present in the droplet, different amplicons can be ligated to different label species. The detection of ROs with multiple species of labels can indicate that more than one species of label was present in the droplet. Such methods can include forming a droplet containing ROs, amplifying the ROs within the droplet to form amplicons, and labeling the amplicons with droplet specific labels. The labeling can be performed by any of the methods described herein.

As such, the detection of the same RO comprising different labels can indicate that more than one label was present in the droplet during the amplification step. This result can indicate the merger of two droplets, each of which contained its own droplet-specific label. Alternatively or in addition, the result can indicate that a single droplet was loaded with two different labels.

The labels can be attached to the ROs using binding sequences contained in the RO. Alternatively, adapters can be ligated to the ROs that allow for the amplification and labeling of the ROs in the droplet. In general, the sample nucleic acids present in the droplet are also labeled with the same droplet-specific label. This can allow the sample nucleic acids and ROs to be identified as originating in a common droplet. As will be discussed herein, it can be useful to identify sample nucleic acids and ROs as originating in a common droplet because the ROs can be used to determine a variety of characteristics of an individual droplet, which can be used to assess the quality of the data obtained from the contents of that individual droplet.

(ii) Providing Labels to Droplets

The methods generally include providing a plurality of labels, such as barcodes, to the droplets. In some such cases, the labels are attached to a solid substrate, sometimes referred to as a barcode bead or barcode ball. Examples of solid substrates include nano- or microparticles. The solid substrate can comprise hydrogels or polymers in some embodiments. Generally, the barcodes attached to the same solid support contain a common sequence. Thus, introducing the solid support to the droplet can include introducing a plurality of barcodes with a common sequence to the droplet.

These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

Unique molecular identifiers (UMIs) can also be added to the molecules on the bead surfaces by, for example, a PCR hybridization and extension with primers that have a random UMI sequence. This would permit every individual barcode on a given bead's surface to have a unique identifier, so that bias in the rates at which different molecules are amplified during generation of a sequencing library can be partly corrected by disregarding and/or aggregating duplicated UMIs in quantitation.

With a hard bead, like a polystyrene bead, most of the oligo synthesis will be confined to the surface of the bead. However, hydrogel beads, like polyacrylamide, agarose, alginate, etc., can also be used, with the advantage that they are porous, permitting the oligos to be synthesized even within the bulk of the beads. These porous beads have the benefit of permitting a much larger number of oligos to be synthesized on/and or in the bead, which may be advantageous for applications that require large numbers of target molecules to be labeled with the barcodes or to control the stoichiometry of the barcode concentration in the subsequent reactions.

Another advantage of hydrogels and other polymer beads is that they can be induced to melt or dissolve by changing environmental conditions. For example, with beads made of low melting point agarose, it is possible to melt the agarose beads in a droplet that is heated above the melting point of the hydrogel, which may happen during thermal cycling for PCR. This has the advantage of allowing the barcodes to mix into the bulk of the droplet, which may enhance the efficiency of the barcoding reaction. Additionally, discrete entities, e.g., droplets, that contain the beads can be sorted based on whether they contain a specific number of beads, such as 0, 1, 2, etc., beads. This is advantageous because it can be used, for example, to generate a plurality of discrete entities in which nearly every discrete entity contains the exact number of desired beads, such as one bead. For example, when barcoding cellular nucleic acids, one bead may be paired with one cell or cell lysate from a single cell in a discrete entity, e.g., a droplet.

Where the encapsulation of cells, such as cells from a sample, is achieved using random encapsulation techniques, only certain discrete entities, e.g., droplets, will contain a single cell while, since the same is true for the beads, only certain discrete entities will contain a single bead. The probability of obtaining a discrete entity that has exactly one cell and one bead then becomes the probability of encapsulating one cell and one bead in the same discrete entity, which can often be low. This can greatly reduce the efficiency of the process that generates the barcoded molecular targets, e.g. cellular nucleic acids. By sorting to ensure that only discrete entities containing a bead are used to encapsulate cells, the efficiency of the pairing can be increased significantly. Such paring can then be verified by the ROs contained within the droplet. Alternatively, the use of ROs can eliminate the need to sort droplets because the presence of more than one barcode bead can be detected from the resulting sequencing data.

Accordingly, in some embodiments the present disclosure provides a method of introducing multiple copies of a nucleic acid barcode sequence into a discrete entity, wherein the method includes: (a) encapsulating a plurality of nucleic acid target molecules in a discrete entity (such as target molecules from a sample and/or ROs); (b) introducing into the discrete entity a porous bead including multiple copies of a nucleic acid barcode sequence, wherein the multiple copies of the nucleic acid barcode sequence are distributed at least in part on surfaces defined by one or more pores of the porous bead; and (c) subjecting the discrete entity to conditions sufficient for enzymatic incorporation of the nucleic acid barcode sequence into the plurality of nucleic acid target molecules or amplification products thereof. This method could also be performed using a non-porous bead, wherein the multiple copies of the nucleic acid barcode sequence are distributed on the surface of the non-porous bead, e g, bound to the non-porous bead via a nucleic acid binding molecule.

In some embodiments, the method includes: (a) encapsulating a plurality of nucleic acid target molecules in a first discrete entity; (b) encapsulating a bead in a second discrete entity, wherein the second discrete entity is a droplet and the bead includes multiple copies of a nucleic acid barcode sequence on a surface thereof, and wherein the step of encapsulating the bead in the second discrete entity includes (i) flowing a plurality of beads through a channel of a microfluidic device, the microfluidic device including a droplet generator in fluid communication with the channel, under conditions sufficient to effect inertial ordering of the beads in the channel, thereby providing periodic injection of the beads into the droplet generator; and (ii) matching the periodicity of the injection with the periodicity of droplet generation of the droplet generator, thereby encapsulating individual beads in individual droplets using the droplet generator; (c) merging the first and second discrete entities; and (d) subjecting the merged discrete entities to conditions sufficient for enzymatic incorporation of the nucleic acid barcode sequence into the plurality of nucleic acid target molecules or amplification products thereof.

In some embodiments, the method includes: (a) encapsulating a plurality of nucleic acid target molecules in a discrete entity; (b) introducing into the discrete entity a bead including multiple copies of a nucleic acid barcode sequence on a surface thereof, wherein each copy of the nucleic acid barcode sequence includes a unique molecular identifier (UMI) attached thereto; and (c) subjecting the discrete entity to conditions sufficient for enzymatic incorporation of the nucleic acid barcode sequence into the plurality of nucleic acid target molecules or amplification products thereof.

In some embodiments, the method includes: (a) encapsulating a plurality of nucleic acid target molecules in a first discrete entity; (b) encapsulating a bead in a second discrete entity, wherein the second discrete entity is a droplet and the bead includes multiple copies of a nucleic acid barcode sequence on a surface thereof, and wherein the step of encapsulating the bead in the second discrete entities includes (i) flowing a plurality of beads through a channel of a microfluidic device, the microfluidic device including a droplet generator in fluid communication with the channel, (ii) encapsulating one or more beads in one or more discrete entities produced by the droplet generator, and (iii) sorting the one or more discrete entities produced by the droplet generator to remove discrete entities which do not include one or more beads; (c) merging the first and second discrete entities; and (d) subjecting the merged discrete entities to conditions sufficient for enzymatic incorporation of the nucleic acid barcode sequence into the plurality of nucleic acid target molecules or amplification products thereof.

Another such method of introducing barcodes into a droplet includes introducing a cell into the droplet, wherein the barcode is expressed in the cell, for example, as a high copy number plasmid. This serves to increase the starting concentration of the barcode so that it can be more easily integrated into the sequences of the cell nucleic acids. A suitable plasmid may be, e.g., from about 1 kb to about 3 kb in size.

Yet another such method includes introducing multiple copies of a nucleic acid barcode into a discrete entity. In one such embodiment, the discrete entity is a droplet. Such droplets can be merged with a droplet containing the ROs and/or sample material. One way to produce barcodes for use in reactions, e.g., reactions occurring in discrete entities, e.g., droplets, is using digital PCR. In this approach, individual DNA barcode sequences are encapsulated in discrete entities at limiting dilution, such that a fraction of discrete entities contain no molecules and, normally, a much smaller fraction contain single molecules. Reagents sufficient for amplification are also included in the discrete entity and the discrete entities incubated under conditions sufficient to induce amplification such as, for example, thermal cycling for PCR. The amplification fills each droplet with many copies of the original molecule. This library can be used directly or, if desired, sorted using active or passive means to discard empty discrete entities.

A library of synthesized barcodes with a random region (for example, or any variation of random bases) can be encapsulated in drops so that most drops contain one or no barcodes. The single barcodes within drops are amplified by using universal sequences as a priming site. Exemplary nucleic acid amplification methods that can be used to amplify the single barcodes include: PCR, strand displacement amplification, rolling circle amplification, helicase dependent isothermal amplification, recombinase based PCR (twistamp), and loop mediated amplification (LAMP).

To use the barcode discrete entity library, the discrete entities in the library can be combined with the molecular targets, e.g., nucleic acids, intended for barcoding and subjected to a barcoding reaction. The benefit of amplifying the barcodes prior to introducing them to the molecular targets is that their concentration can be greatly increased, making the subsequent barcoding reactions more efficient in some instances. For example, with an unamplified barcode, many cycles of PCR may be necessary to amplify the barcode and then allow its attachment to target nucleic acids when using a splicing by overlap extension approach. This large amount of amplification can degrade reagents before linkage occurs, resulting in inefficiency, and also necessitate additional thermal cycling, which can produce amplification bias. In addition to PCR, which requires thermal cycling, isothermal methods can also be used, such as, for example Loop-mediated isothermal amplification (LAMP), multiple displacement amplification (MDA), multiple annealing and looping-based amplification cycles (MALBAC), etc.

The discrete entities, e.g., droplets, containing the barcodes can also be solidified, generating gel particles filled with barcode molecules. The molecules can be attached to the gels using covalent or non-covalent interactions, permitting the gel beads to be dispersed in an aqueous solvent, or attached to the surface of a bead in the discrete entity.

Accordingly, in some embodiments the present disclosure provides a method of introducing multiple copies of a nucleic acid barcode sequence into a discrete entity, wherein the method includes: (a) encapsulating individual nucleic acid barcode sequences in a population of discrete entities at limiting dilution such that each individual discrete entity of the population of discrete entities statistically contains either zero or one nucleic acid barcode sequence; (b) enzymatically amplifying the nucleic acid barcode sequences in the population of discrete entities to provide a plurality of discrete entities wherein each discrete entity of the plurality of discrete entities includes multiple copies of the individual nucleic acid barcode sequence for that discrete entity; (c) introducing into one or more of the plurality of discrete entities a plurality of nucleic acid target molecules; and (d) subjecting the one or more of the plurality of discrete entities to conditions sufficient for enzymatic incorporation of the nucleic acid barcode sequence into the plurality of nucleic acid target molecules or amplification products thereof.

In another such method, the multiple copies of the nucleic acid barcode can be added to a droplet by pico-injection or droplet/stream merger.

(iii) Attaching Labels to Nucleic Acid Targets

The present disclosure provides a variety of methods for the attachment of nucleic acid labels to nucleic acid target molecules and/or amplification products thereof. As discussed above, the labels frequently comprise barcodes. The target molecules are generally the ROs and/or sample nucleic acids, such as nucleic acids from a sample, such as a cell. The target molecules are generally contained within a droplet. The labels include any of the labels described herein. The processes of attaching labels to nucleic acids generally take place in a discrete entity, such as a droplet. Labels can be provided to the droplets using the methods described above.

One objective of the barcoding strategy of this disclosure is to enable independent sequence reads to be associated with one another via a barcode. The barcode identifies reads that originated from molecules that existed within the same discrete entity, such as a droplet. The molecules contained within the same droplet are generally labeled with the same barcode. Important to this concept is a methodology for attaching barcodes to target nucleic acids in a droplet. There are numerous techniques that can be used to attach barcodes to the nucleic acids within a discrete entity.

Some of the methods described herein involve first amplifying the target molecules, which can include amplifying the sample nucleic acid molecules and/or the ROs. In some of such methods, the labels can be ligated to the resulting amplicons. In some cases, the method can include ligating adapters to the amplicons, which adapters can contain the label and another segment, such as a primer binding site.

In some embodiments, the labels are added using adaptors present on the target nucleic acids. The adaptor sequences can be added to the target nucleic acids by ligation. In some instances, the ROs contain the adaptor sequences in the region common to a plurality of the ROs, as described above. The adaptor sequences can include a known sequence to which primers can be bound.

The barcodes can then be attached to the target molecules using, for example, splicing by overlap extension. In this example, the barcodes and the target molecules comprise adapter sequences such that the amplicons produced by an amplification reaction can anneal to each other. The annealed target molecule and barcode are then extended onto one another via an extension reaction, such as DNA polymerization. This generates a double-stranded product that contains the target nucleic acids attached to the barcode sequences.

Accordingly, in some embodiments the present disclosure provides a method for barcoding nucleic acid target molecules, wherein the method includes: (a) introducing into a discrete entity (i) a nucleic acid target molecule, (ii) a nucleic acid barcode sequence, (iii) a first set of primers configured to amplify a sequence of the nucleic acid target molecule, (iv) a second set of primers configured to amplify a sequence of the nucleic acid barcode sequence, wherein one of the first set of primers includes a sequence which is at least partially complementary to a sequence of one of the second set of primers, and (v) an enzymatic amplification reagent; (b) subjecting the discrete entity to conditions sufficient for enzymatic amplification of a sequence of the nucleic acid target molecule and a sequence of the nucleic acid barcode sequence, wherein amplification products having regions of partial sequence homology are produced; and (c) subjecting the discrete entity to conditions sufficient for complementary regions of sequences of the amplification products to hybridize and for the hybridized sequences to be enzymatically extended, thereby providing a product including the amplified sequence of the nucleic acid target molecule and the amplified sequence of the nucleic acid barcode sequence. In some embodiments, the target molecule is an RO. Alternatively or in addition, the target molecule can be a polynucleotide derived from a sample, such as a cell.

In another example, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, the adaptor sequences.

An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule. In some embodiments, the ROs and sample nucleic acids are amplified prior to barcoding.

Accordingly, the present disclosure provides a method for barcoding nucleic acid target molecules, wherein the method includes: (a) introducing into a discrete entity (i) a plurality of nucleic acid target molecules, (ii) a plurality of nucleic acid barcodes and (iii) an enzymatic amplification reagent; (b) subjecting the discrete entity to conditions sufficient for enzymatic amplification of sequences of the plurality of nucleic acid target molecules and sequences of the plurality of nucleic acid barcodes, wherein amplification products having regions of partial sequence homology are produced; and (c) subjecting the discrete entity to conditions sufficient for complementary regions of sequences of the amplification products to hybridize and for the hybridized sequences to be enzymatically extended, thereby providing a plurality of products, each including an amplified sequence of one of the plurality of target nucleic molecules and an amplified sequences of one of the plurality of nucleic acid barcodes. In some embodiments, the plurality of nucleic acid target molecules comprises one or more ROs.

In other embodiments, the present disclosure provides a method for barcoding nucleic acid target molecules, wherein the method includes: (a) generating a library of nucleic acid barcode primers, wherein each nucleic acid barcode primer in the library includes a first sequence sufficient to anneal to a nucleic acid target molecule and a second sequence including a nucleic acid barcode sequence; (b) combining one or more nucleic acid barcode primers selected from the library and one or more nucleic acid target molecules in each of a plurality of discrete entities, wherein the one or more primers selected from the library for inclusion in each discrete entity includes one or more primers with a first sequence sufficient to anneal to one or more of the nucleic acid target molecules in that discrete entity; and (c) enzymatically amplifying one or more of the nucleic acid target molecules in each discrete entity using one or more of the nucleic acid barcode primers in that discrete entity, such that amplification products including a sequence of one of the one or more nucleic acid target molecules and a nucleic acid barcode sequence are produced. In some embodiments, at least one of the one or more nucleic acid target molecules is an RO.

In other embodiments, the present disclosure provides a method for barcoding nucleic acid target molecules, wherein the method includes: (a) generating a library of nucleic acid barcode sequences; (b) combining in each of a plurality of discrete entities one or more nucleic acid barcode sequences selected from the library and one or more nucleic acid target molecules; and (c) enzymatically fragmenting the one or more nucleic acid target molecules in each discrete entity and enzymatically incorporating one or more of the one or more nucleic acid barcode sequences in each discrete entity into fragments of the one or more target nucleic acid molecules or amplification products thereof in that discrete entity.

In other embodiments, the present disclosure provides a method for barcoding nucleic acid target molecules, wherein the method includes: (a) generating a library of nucleic acid barcode sequences; (b) combining in each of a plurality of discrete entities one or more nucleic acid barcode sequences selected from the library and one or more nucleic acid target molecules; and (c) enzymatically ligating the one or more nucleic acid target molecules in each discrete entity to one or more nucleic acid barcode sequences in that discrete entity. In some embodiments, the target molecules are amplified prior to the ligating. In some embodiments, the target molecules comprise one or more ROs.

In some aspects, the methods can also include RT-PCR. RT-PCR can be performed in drops to attach barcodes to cDNA and amplify the linked products.

B. Sequencing

The methods typically include sequencing the target nucleic acids, which nucleic acids can include the ROs and/or nucleic acids derived from a sample. Sequencing generally occurs after the target nucleic acids have been labeled, if necessary. In some embodiments, the methods of the present disclosure can be used to sequence target nucleic acids, including ROs and nucleic acids derived from samples, such as cells. The target nucleic acids can include single molecules originating from the nucleic acids of single cells and/or ROs containing unique sequences. To accomplish this, it may often be desirable to amplify the molecules so that there are multiple copies of each molecule in the droplet. Such amplification can take place either before or after the molecules are labeled. Amplification permits multifold sequencing of each original molecule, which can enable the collection of accurate data that reduces source error. Amplification is described above.

In some cases, the methods disclosed herein are used in combination with an existing sequencing technology. In further cases, the methods disclosed herein are used with technologies and approaches derived from any existing sequencing technology. Cases of sequencing technologies that can be used with the methods disclosed herein include, but are not limited to, the Illumina® sequencing-by-synthesis platform (Illumina, San Diego, Calif.), the SOLiD™ system (Applied Biosystems Corp.), pyrosequencing (e.g., 454 Life Sciences, subsidiary of Roche Diagnostics), a sequencing technique based on semiconductor detectors (e.g., the Ion Torrent® platform), nanopore sequencing (e.g., the Oxford Nanopore sequencing platform), DNA nanoball sequencing methods (e.g. Complete Genomics), sequencing by hybridization and any other suitable technology, or any technology that may be derived from any of the above technologies.

In some embodiments, read pairs comprise two distinct sequences of a target nucleic acid sample. In some embodiments, a read pair comprises a sequence read of a target nucleic acid sample in combination with a sequence read of a molecular tag, such that all target nucleic acid sample reads corresponding to a common molecular tag sequence map to the same droplet. In some aspects, reads or read pairs with the same molecular tag map to the same nucleic acid molecule within a target nucleic acid sample. Accordingly, in some embodiments molecular tag sequence is used to sort target nucleic acid sample reads into “tagged bins,” which in some embodiments each correspond to a single droplet and/or a single molecule of a target nucleic acid sample.

C. Determining Characteristics of a Droplet from Sequencing Data

Sequencing information obtained from ROs present in a droplet can be used to assess a variety of characteristics of that droplet. Because the methods often employ the use of fluids containing ROs at a fixed starting concentration, the number of RO species matched to a particular barcode can roughly correlate with the volume of fluid added to each drop. Drops often begin the methods monodisperse and roughly uniform in size. As such, the number of RO species obtained from the final drop can indicate the number of monodisperse drops that merged to make that final drop. The number of RO species can also be used to determine the size of drops of various volumes, such as those created in polydisperse, shaken emulsions, or in emulsions in which mergers were not expected to occur.

In some of such cases, a method of evaluating a quality characteristic of a droplet can include encapsulating a plurality of polynucleotides in a droplet, wherein the plurality of polynucleotides comprises at least one species of oligonucleotide; tagging the polynucleotides with a label that identifies the polynucleotides as arising from the droplet; counting a number of species of oligonucleotide tagged with the label; and determining a quality characteristic of the droplet based on the number of species of oligonucleotide tagged with the label.

A single stable droplet containing a single set of unique RO species and single set of droplet-specific barcodes should yield a population of ROs, each labeled with the droplet-specific barcode. Thus, the detection of an RO species labeled with more than one barcode can be informative of a variety of possible events. In one example, the detection of an RO species associated with barcodes associated with more than one drop can indicate droplet merger. Such a merger can involve the merger of two drops, each drop containing its own droplet specific barcode. The merged drop that results, therefore, may contain two different sets of barcodes instead of one. If the RO is amplified and copies are found associated with more than one barcode, this could be due to droplet merger. Alternatively or in addition, if the RO is amplified and copies are found associated with more than one barcode, this could be due to the presence of more than one species of barcode being loaded into the droplet.

In some of such cases, a method of evaluating a quality characteristic of a droplet can include sequencing a plurality of polynucleotides obtained from the droplet, wherein the plurality of polynucleotides comprises at least one oligonucleotide species comprising a first nucleic acid segment and a second nucleic acid segment, wherein the first nucleic acid segment comprises a plurality of random nucleotides; and the second nucleic acid segment comprises a conserved region comprising a label, detecting sequences of oligonucleotide species comprising labels; determining a quality characteristic of the droplet based on the sequences of the oligonucleotide species detected. In some cases, the plurality of polynucleotides comprises a first oligonucleotide species comprising a first conserved region and second oligonucleotide species comprising a second conserved region, wherein the detecting of sequences encoding the first oligonucleotide species and second oligonucleotide species is informative of a droplet merger.

In various cases, the first oligonucleotide species comprises a first label indicative of a first group of droplets and a droplet-specific label and the second oligonucleotide species comprises a second label indicative of a second group of droplets and the droplet specific label.

Alternatively or in addition, the plurality of polynucleotides comprises plurality of polynucleotides encoding a first oligonucleotide species, wherein at least one member of the plurality of nucleotides encoding the first nucleotide species is labeled with a first label and at least one member of the plurality of nucleotides encoding the first nucleotide species is labeled with a second label.

The pairing of the barcode tag and the RO can also be used to assess barcoding fidelity, such that the contents of one drop are tagged with exactly one barcode, or in alternate embodiments regular intervals of barcodes such as whole number or known fraction intervals of barcodes such that droplet volume is approximated. Each droplet partition generally receives only one barcode or a predictable number of categories of barcodes and an expected number of instances of the synthetic sequences, such that a cell or droplet source can be identified and its volume confidently assessed. If the expected number of synthetic sequences for a single droplet is associated with multiple barcoding tags or a higher diversity of tags than expected, this could mean that an error occurred during synthesis of the barcoding tags and that the barcoding fidelity is compromised. As another example, the association of a single droplet with multiple barcoding tags can indicate a problem with attaching a single barcode species to a single solid support.

As another exemplary use, ROs can be used to detect the merging of two or more sets of different drop populations. In some of the methods described herein, individual droplets or subpopulations of droplets can be loaded with their own distinguishable set of ROs. These ROs can contain a combination of random nucleic acids and conserved nucleic acids. The conserved nucleic acids can be held in common between the ROs of the set and different between ROs of different sets. This can identify the ROs as belonging to the same set. In such methods, a first droplet containing a first set of ROs and a second droplet containing a second set of ROs can be merged. The ROs from the first and second sets can be labeled with the same droplet-specific barcode. Sequencing and counting the number of ROs from each set can be informative of the volume of each droplet used to make the merged droplet. Furthermore, detecting sequences from both sets of droplets with the same barcode can confirm that the merger between the first and second droplets took place. Such methods can be useful, for example, when merging a first droplet containing a sample with a second droplet containing reagents.

In some embodiments, the present disclosure provides a method of characterizing the number of ROs present in a droplet. For example, in some embodiments, the present disclosure provides a method for characterizing the number of ROs present in a droplet, wherein the method includes (a) isolating at least one RO in a droplet; (b) incorporating unique molecular identifiers (UMIs) into the at least one RO; (c) sequencing the at least one RO; and (e) using the sequence of the RO to infer the number of species of ROs in the droplet.

In some embodiments, the present disclosure provides a method for characterizing the number of ROs present in each drop in a plurality of droplets, wherein the method includes (a) isolating at least one RO in each of the plurality of droplets; (b) incorporating unique molecular identifiers (UMI)s into the at least one RO, wherein the sequence of the UMI is common to ROs contained within the same droplet and different between ROs contained within different droplets; (c) sequencing the at least one RO; and (e) using the sequence of the RO and the UMI to infer the number of species of ROs in each droplet.

For an RO concentration where the average number per unit drop size is A, the likelihood of counting n ROs can be, for example:

$$P ( n \vert λ ) = \frac{ {λ}^{n} {e}^{- λ}}{n !} \qquad ( Formula Ι )$$

This distribution has a mean at A and a standard deviation of \sqrt{λ}. In drops that are the size of m unit drops, one may expect a new distribution where λ→mλ. In such cases, the sum of Poisson distributions (as in the case of mergers) can also follow a Poisson distribution wherein the new mean is the sum of the constituent means. This new m-fold distribution thus can have a mean occupation is mλ and a standard deviation is

$$P ( n \vert m λ ) = \frac{{( m λ )}^{n} {e}^{- m λ}}{n !} \qquad ( Formula ΙΙ )$$

As λ grows, the standard deviation (the peak width) can decrease relative to the mean such that distributions can tighten and can be easier to distinguish. For example, the central limit theorem implies that for large λ or large m, a Poisson distribution approaches a normal distribution with the corresponding means and standard deviations.

For an RO concentration where the average number per unit drop size is λ, the likelihood of counting n ROs can be, for example:

$$P ( n \vert λ ) = \frac{{λ}^{n} {e}^{- λ}}{n !} \qquad ( Formula Ι )$$

This distribution has a mean at A and a standard deviation of \sqrt{λ}. In drops that are the size of m unit drops, one may expect a new distribution where λ→mλ. In such cases, the sum of Poisson distributions (as in the case of mergers) can also follow a Poisson distribution wherein the new mean is the sum of the constituent means. This new m-fold distribution thus can have a mean occupation is mA and a standard deviation is \sqrt{mλ}.

$$P ( n \vert m λ ) = \frac{{( m λ )}^{n} {e}^{- m λ}}{n !} \qquad ( Formula ΙΙ )$$

As λ grows, the standard deviation (the peak width) can decrease relative to the mean such that distributions can tighten and can be easier to distinguish. For example, the central limit theorem implies that for large λ or large m, a Poisson distribution approaches a normal distribution with the corresponding means and standard deviations.

An additional aspect of some methods disclosed herein is how to determine a drop's size based on its measured occupancy and the average unit occupancy. One exemplary method includes setting strict ranges within which anything is counted towards a certain size. This is depicted in FIG. 3a, which plots several distributions for possible occupancies given a drop of size m (“actual”, blue) and unit occupancy λ. For those distributions, a drop is called a size m (“detected”, red) if it falls in the range [(m−½)λ, (m+½)λ]. That range is demarcated with a dotted line.

Some embodiments of this protocol can be improved when there is a strong correlation between drop volume and RO count. ROs, like other reagents, are generally loaded into droplets following Poisson statistics when there is a uniform or generally consistent concentration of non-interacting ROs being partitioned into droplets.

The synthetic oligonucleotides often comprise constant regions required for association with the barcoding tag and subsequent PCR amplification. The oligonucleotides can be single or double stranded, DNA, RNA, mixed DNA/RNA molecules, including molecules containing standard nucleotides, modified nucleotides, or both. Depending on the approach used to pair barcode tags and the randomized synthetic oligonucleotides, single or double stranded versions of the oligonucleotides are often preferable.

In some embodiments, the present disclosure provides a method for detecting the merger of two drops, each drop containing its own drop-specific label. As previously described, some of the methods herein include a step of amplifying the ROs. Such amplification can take place either before or after the ROs are labeled. In such cases, individual amplicons of the same RO species can be labeled with the different barcodes contained within the same drop. Detecting sequences of the same RO species with different barcodes can therefore indicate the merger of more than one droplet. In such cases, it is possible that the merged droplet contains samples from each of the original droplets.

Detecting the merger of two droplets can allow sequences derived from the samples to be further evaluated or excluded from downstream analysis because such sequences can contain the same barcodes as the ROs used to detect the merger. This can be important, for example, when droplets are intended to contain single cells. The exclusion of merged droplets containing more than one cell can help retain single-cell resolution in the resulting sequencing data.

Accordingly, in some embodiments the methods disclosed herein comprise detecting a merger between at least two droplets, the method comprising (a) isolating at least one RO in each of a plurality of droplets; (b) incorporating at least one unique molecular identifier (UMI) into at least one copy of the at least one RO, wherein the sequence of the UMI is common to ROs contained within the same droplet and different between ROs contained within different droplets; (c) sequencing the at least one RO; and (e) detecting a plurality of copies of the at least one RO, wherein a first copy of the RO contains a first UMI and a second copy of the RO contains a second UMI.

In some embodiments, the present disclosure provides a method for detecting the merger of two drops, each drop containing its own class of RO. As previously described, some ROs can contain a unique region and at least one region common to a plurality of ROs. In some cases, the plurality of ROs can be a class or subpopulation of ROs. Thus, different populations of ROs can be used in different fluids to create different populations of droplets. As an example, a first population of ROs can be loaded into a first fluid containing a sample and a second population of ROs can be loaded into a second fluid containing reagents. Populations of droplets can be formed from each fluid, and the resulting droplets can be merged. The contents of the merged droplet can be labeled in a droplet-specific manner and sequenced. The sequencing data can be used to determine a variety of characteristics, including, for example, the volume of each original droplet and confirmation that the droplets from different populations merged. The sequencing data can also be used to determine if more than one droplet merged unintentionally, for example, with another droplet of the same population. In such cases, the sequencing data may reveal that the same species of ROs from the first and second populations of ROs, which originated from the first and second fluids used to make the first and second populations of droplets, respectively, are labeled with more than one droplet-specific label. This result can indicate the merger of more than the intended number of drops.

### 3. Biological Samples

The biological samples are variously derived from non-cellular entities comprising polynucleotides (e.g., a virus) or from cell-based organisms (e.g., member of archaea, bacteria, or eukarya domains). The biological sample can be a blood sample. The biological sample can be a cell sample such as a cell culture sample. Cell culture samples include cells in suspension or adherent cells that are lifted from a cell culture dish (e.g., by trypsinization). Cell culture samples can be derived from primary cells or cells from an established cell line, among others.

The biological sample is often derived or obtained from a subject, e.g., plants, fungi, eubacteria, archeabacteria, protists, or animals. The subject is often an organism, either a single-celled or multi-cellular organism. The biological sample is isolated initially from a multi-cellular organism in any suitable form. The animal is sometimes a fish, e.g., a zebrafish. The animal is sometimes a mammal. The mammal is sometimes, without limitation, a dog, cat, horse, cow, mouse, rat, or pig. The mammal is sometimes, without limitation, a primate, e.g., a human, chimpanzee, orangutan, or gorilla. The human is male or female. The sample is sometimes derived from a human embryo or human fetus. The human is an infant, child, teenager, adult, or elderly person. The female is sometimes pregnant, suspected of being pregnant, or planning to become pregnant. The sample is sometimes a single or individual cell from a subject and the biological molecules are derived from the single or individual cell. The sample is sometimes an individual micro-organism, or a population of micro-organisms, or a mixture of microorganisms and host cellular or cell free nucleic acids.

The biological sample is often obtained from a subject (e.g., human subject) who is healthy. The biological sample is sometimes obtained from a subject (e.g., an expectant mother) at least 4, 5, 6, 7, 8, 9, 10, 11, 12, 13, 14, 15, 16, 17, 18, 19, 20, 21, 22, 23, 24, 25, or 26 weeks of gestation. Sometimes, the subject is affected by a genetic disease, is a carrier for a genetic disease or is at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs).

The biological sample is sometimes from a subject who has a specific disease, disorder, or condition, or is suspected of having (or at risk of having) a specific disease, disorder or condition. For example, the biological sample is from a cancer patient, a patient suspected of having cancer, or a patient at risk of having cancer. The cancer is, without limitation, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample is often derived from the cancer and/or normal tissue from the cancer patient. The biological sample sometimes is biopsy of a tumor. Alternately, the biological sample is a blood sample that comprises circulating tumor cells (CTCs).

The biological sample is sometimes derived from and includes a variety of sources, including, without limitation, aqueous humour, vitreous humour, bile, whole blood, blood serum, blood plasma, breast milk, cerebrospinal fluid, cerumen, enolymph, perilymph, gastric juice, mucus, peritoneal fluid, saliva, sebum, semen, sweat, tears, vaginal secretion, vomit, feces, or urine. The biological sample is sometimes obtained from a hospital, laboratory, clinical or medical laboratory. The sample is often taken from a subject.

Often, the biological sample is an environmental sample comprising medium such as water, soil, air, and the like. The biological sample is sometimes a forensic sample (e.g., hair, blood, semen, saliva, etc.). The biological sample is sometimes an agent used in a bioterrorist attack (e.g., influenza, anthrax, smallpox).

The biological sample is often processed to render it competent for performing any of the methods provided herein. For example, the biological sample is dissociated to generate a dissociated cell population. Biological cells or entities are often encapsulated in droplets prior to further processing, in accordance with the methods provided herein. Droplets often contain, on average, no more than a single biological cell or entity. A single biological cell or entity is sometimes lysed or otherwise disrupted within a droplet. Methods of lysing biological cells within droplets consistent with the methods and compositions described herein are described in the art.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### 4. Numbered Embodiments and Figures

Provided herein are methods and compositions for evaluating emulsion uniformity. 1. A method of evaluating a quality characteristic of a droplet comprising: encapsulating a plurality of polynucleotides in a droplet, wherein the plurality of polynucleotides comprises at least one species of oligonucleotide; tagging the polynucleotides with a label that identifies the polynucleotides as arising from the droplet; counting a number of species of oligonucleotide tagged with the label; and determining a quality characteristic of the droplet based on the number of species of oligonucleotide tagged with the label. 2. The method of embodiment 1, wherein an oligonucleotide comprises: a first nucleic acid segment and a second nucleic acid segment, wherein the first nucleic acid segment comprises a plurality of random nucleotides; and the second nucleic acid segment comprises a conserved region common to the plurality of oligonucleotides. 3. The method of embodiment 1 or embodiment 2, further comprising forming the droplet in a microfluidic device. 4. The method of embodiment 3, wherein forming the droplet comprises isolating a portion of a first fluid, wherein the first fluid comprises a known concentration of oligonucleotide species. 5. The method of embodiment 4, wherein the first fluid comprises a plurality of the species of oligonucleotide dispersed throughout the first fluid, and wherein isolating the portion of the first fluid comprises isolating a number of oligonucleotide species in the droplet according to a Poisson distribution that is proportional to the volume of the droplet. 6. The method of any one of embodiments 1-5, wherein the droplet comprises an aqueous phase fluid dispersed in an immiscible phase carrier fluid. 7. The method of any one of embodiments 1-6, wherein the droplet comprises a known concentration of oligonucleotides. 8. The method of any one of embodiments 1-7, wherein the plurality of polynucleotides comprises a number of polynucleotides encapsulated according to a Poisson distribution dependent on a volume of the droplet. 9. The method of any one of embodiments 1-7, wherein the number of species of oligonucleotide tagged with the label is informative of a volume of the droplet. 10. The method of embodiment 9, wherein the volume of the droplet comprises a volume of the immiscible phase carrier fluid. 11. The method of any one of embodiments 1-10, wherein the plurality of polynucleotides further comprises polynucleotides obtained from a sample. 12. The method of embodiment 11, wherein the sample comprises a cell. 13. The method of embodiment 12, wherein the sample comprises no more than one cell. 14. The method of any one of embodiments 1-13, wherein encapsulating the plurality of polynucleotides in the droplet comprises encapsulating a cell comprising polynucleotides in the droplet. 15. The method of embodiment 14, further comprising lysing the cell in the droplet. 16. The method of any one of embodiments 1-15, wherein the label comprises a barcode. 17. The method of any one of embodiments 1-16, wherein tagging the polynucleotides with a label comprises subjecting the droplet to conditions sufficient for enzymatic incorporation of the label into the plurality of polynucleotides. 18. The method of embodiment 17, wherein enzymatic incorporation of the label into the plurality of polynucleotides comprises ligating the label to the polynucleotides. 19. The method of any one of embodiments 1-18, wherein tagging the polynucleotides with a label comprises subjecting the droplet to conditions sufficient for enzymatic incorporation of the label into amplification products of the plurality of polynucleotides. 20. The method of embodiment 19, wherein enzymatic incorporation of the label into amplification products of the plurality of polynucleotides comprises ligating the label to the amplification products. 21. The method of embodiment 19, wherein enzymatic incorporation of the label into amplification products of the plurality of polynucleotides comprises amplifying the plurality of polynucleotides by PCR using barcoded primers. 22. The method of any one of embodiments 1-21, wherein counting a number of species of oligonucleotide tagged with the label comprises sequencing the polynucleotides tagged with the label. 23. The method of any one of embodiments 1-22, wherein the quality control characteristic is a volume of the droplet. 24. The method of any one of embodiments 1-23, wherein the quality control characteristic is a merger of two droplets. 25. The method of any one of embodiments 1-24, wherein data arising from the droplet is adjusted based on the quality characteristic. 26. The method of embodiment 25, wherein the quality characteristic comprises a volume of the droplet. 27. The method of embodiment 25 or embodiment 26, wherein the quality characteristic comprises a droplet merger. 28. The method of any one of embodiments 1-24, wherein data arising from the droplet is excluded from further analysis based on the quality characteristic. 29. The method of embodiment 28, wherein the quality characteristic comprises a volume of the droplet. 30. The method of embodiment 28 or embodiment 29, wherein the quality characteristic comprises a droplet merger.

31. A method of evaluating a quality characteristic of a droplet comprising: sequencing a plurality of polynucleotides obtained from the droplet, wherein the plurality of polynucleotides comprises at least one oligonucleotide species comprising a first nucleic acid segment and a second nucleic acid segment, wherein the first nucleic acid segment comprises a plurality of random nucleotides; and the second nucleic acid segment comprises a conserved region comprising a label, detecting sequences of oligonucleotide species comprising labels; and determining a quality characteristic of the droplet based on the sequences of the oligonucleotide species detected. 32. The method of embodiment 31, wherein the plurality of polynucleotides comprises a first oligonucleotide species comprising a first conserved region and second oligonucleotide species comprising a second conserved region, wherein the detecting of sequences encoding the first oligonucleotide species and second oligonucleotide species is informative of a droplet merger. 33. The method of embodiment 32, wherein the first conserved region comprises a first label and the second conserved region comprises a second label. 34. The method of embodiment 33, wherein the first label comprises a first barcode and the second label comprises a second barcode. 35. The method of embodiment 32, wherein the first oligonucleotide species comprises a first label indicative of a first group of droplets and a droplet-specific label and the second oligonucleotide species comprises a second label indicative of a second group of droplets and the droplet specific label. 36. The method of embodiment 35, wherein the quality characteristic is a merger between a droplet from the first group of droplets and a droplet from the second group of droplets. 37. The method of any one of embodiments 31-34, wherein the plurality of polynucleotides further comprises polynucleotides obtained from a sample. 38. The method of embodiment 37, wherein the sample comprises a cell. 39. The method of any one of embodiments 32-38, wherein the droplet is a merged droplet, and wherein the method further comprises an intentional merger between a first droplet comprising the first oligonucleotide species and a second droplet comprising the second oligonucleotide species to form the merged droplet. 40. The method of embodiment 39, wherein the first droplet comprises polynucleotides obtained from a sample and the second droplet comprises reagents. 41. The method of embodiment 39, wherein the first droplet comprises polynucleotides obtained from a first sample and the second droplet comprises polynucleotides obtained from a second sample. 42. The method of any one of embodiments 32-38, wherein the droplet is a merged droplet resulting from an unintentional merger between a first droplet comprising the first oligonucleotide species and a second droplet comprising the second oligonucleotide species to form the merged droplet. 43. The method of embodiment 31, wherein the plurality of polynucleotides comprises plurality of polynucleotides encoding a first oligonucleotide species, wherein at least one member of the plurality of nucleotides encoding the first nucleotide species is labeled with a first label and at least one member of the plurality of nucleotides encoding the first nucleotide species is labeled with a second label. 44. The method of embodiment 43, wherein detecting at least one member of the plurality of nucleotides encoding the first nucleotide species labeled with the first label and at least one member of the plurality of nucleotides encoding the first nucleotide species labeled with the second label is informative of droplet merger. 45. The method of embodiment 43 or embodiment 44, wherein the droplet is a merged droplet and wherein the first label is informative of a first droplet and the second label is information of a second droplet, and wherein the first droplet and the second droplet merged to form the merged droplet. 46. The method of any one of embodiments 43-45, where the droplet merger is unintentional. 47. The method of any one of embodiments 43-46, wherein at least one of the first label and second label comprises a barcode. 48. The method of embodiment 42 or 45-47, wherein the first droplet comprises polynucleotides obtained from a first sample and the second droplet comprises polynucleotides obtained from a second sample. 49. The method of any one of embodiments 31-48, wherein data arising from the droplet is adjusted based on the quality characteristic. 50. The method of embodiment 49, wherein the quality characteristic comprises a volume of the droplet. 51. The method of any one of embodiments 49, wherein the quality characteristic comprises a droplet merger. 52. The method of any one of embodiments 31-51, wherein data arising from the droplet is excluded from further analysis based on the quality characteristic. 53. The method of embodiment 52, wherein the quality characteristic comprises a volume of the droplet. 54. The method of any one of embodiments 52, wherein the quality characteristic comprises a droplet merger.

55. A composition comprising a droplet comprising at least one oligonucleotide species comprising a first nucleic acid segment and a second nucleic acid segment, wherein the first nucleic acid segment comprises a non-conserved region; and the second nucleic acid segment comprises a conserved region, and wherein a number of oligonucleotide species present in the droplet is indicative of a quality control characteristic of the droplet. 56. The composition of embodiment 55, wherein the non-conserved region comprises a plurality of random nucleotides. 57. The composition of embodiment 55 or embodiment 56, wherein the conserved region comprises a primer binding site. 58. The composition of any one of embodiments 55-57, wherein the droplet further comprises a plurality of first primers, wherein each primer comprises a region that is complementary to the primer binding site. 59. The composition of embodiment 58, wherein each first primer further comprises a barcode that is common to the plurality of first primers. 60. The composition of any one of embodiments 55-59, wherein the oligonucleotide species further comprises a barcode common to the oligonucleotide species present in the droplet. 61. The composition of any one of embodiments 55-60, wherein the droplet comprises an aqueous droplet. 62. The composition of embodiment 61, wherein the aqueous droplet is surrounded by an oil. 63. The composition of any one of embodiments 55-62, wherein the droplet further comprises a DNA polymerase. 64. The composition of any one of embodiments 55-63, wherein the droplet further comprises a reverse transcriptase. 65. The composition of any one of embodiments 55-64, wherein the quality control characteristic comprises detecting a merger of two droplets. 66. The composition of any one of embodiments 55-65, wherein the quality control characteristic comprises a volume of the droplet.

Turning to the figures, one sees the following: In FIG. 1, one sees four different events, the data readout from the drop, and the inferred origin of the drop. In (a), a drop is generated with an estimated mean of 10 ROs per drop. The readout indicates that there are 12 RO species present in the drop (R1-R12). Thus, the inferred origin is a single intact droplet that received 12 RO species according to a Poisson distribution. In (b), drops are generated with an estimated mean of 10 ROs per drop. The readout indicates that there are 30 RO species (R1-R30) present in a single drop. Thus, the inferred origin of the final droplet containing 30 RO species is a merger of three different drops, each of which contained 10 RO species. The first origin drop contained species R1-R10, the second drop contained R11-R20, and the third drop contained R21-R30. In (c), a drop is generated as the merger of two different types of drops. The readout indicates that the drop contains RO species R1-R10 and S1-S10. Thus, the inferred origin of the final droplet is a merger between a first droplet containing species R1-R10 and a second droplet containing species S1-S10. In (d), a drop is generated by merging three different types of drops, each of which was generated with a mean of 10 type-specific RO species. The readout shows the final droplet contained RO species R1-R20, S1-S10, and T1-T10. The inferred origin of the final droplet is the merger of two R-type droplets with one S-type droplet and one T-type droplet. Thus, the four original droplets contained the following species, respectively: R1-R10, R11-R20, S1-S10, and T1-T10.

In FIG. 2A, one can see that the probabilities of finding a drop with 7 ROs when the mean number of ROs is 3. The X axis shows the number of ROs loaded in a drop between 0 and 12 in increments of 1. The Y axis shows the probability (%) between 0% and 26% in increments of 2%. The line peaking on the left shows the probability of a drop being loaded with a specific number of ROs given a mean of 3 ROs per drop. The probability peaks at 2-3 ROs per drop. There is approximately a 2% change that a drop detected with 7 ROs is the result of over-packing a single unit drop given a mean of 3 ROs per drop. This is depicted in the shaded area at the top as “Unmerged,” which is a droplet containing RO species R1-R7. The line peaking on the right shows the probability of a drop being loaded with a specific number of ROs given a merger of three drops, each of which is from a population with a mean of 3 ROs per drop. The probability peaks at between 8-9 ROs per drop. The image shows that there is approximately a 12% probability that a drop formed by merging three drops from the aforementioned population will contain 7 ROs. This is depicted in the shaded region at the top as the merger of a first droplet containing 1 RO (R1), a second droplet containing 2 ROs (R2 and R3), and a third droplet containing four ROs (R4, R5, R6, and R7) to produce a merged droplet with ROs R1-R7. The probabilities of the unit first, second, and third drops that merged are also plotted. The probability of forming drop with a single RO is approximately 15%, two ROs is approximately 22%, and four ROs is approximately 17%.

In FIG. 2B, one can see that the probabilities of finding a drop with 28 ROs is higher when the mean number of ROs per drop is 10. The X axis shows the number of ROs loaded in a drop between 0 and 40 in increments of 4. The Y axis shows the probability (%) between 0% and 14% in increments of 2%. The line peaking on the left shows the probability of a drop being loaded with a specific number of ROs given a mean of 10 ROs per drop. The probability peaks at between 8-12 ROs per drop. There is approximately a 0% change that a drop detected with 28 ROs is the result of over-packing a single unit drop given a mean of 10 ROs per drop. This is depicted in the shaded area at the top as “Unmerged,” which is a droplet containing RO species R1-R28. The line peaking on the right shows the probability of a drop being loaded with a specific number of ROs given a merger of three drops, each of which is from a population with a mean of 10 ROs per drop. The probability peaks at between 28-32 ROs per drop. The image shows that there is approximately a 7% probability that a drop formed by merging three drops from the aforementioned population will contain 28 ROs. This is depicted in the shaded region at the top as the merger of a first droplet containing 6 ROs (R1-R6), a second droplet containing 9 ROs (R7-R15), and a third droplet containing 13 ROs (R16-R28) to produce a merged droplet with ROs R1-R28. The probabilities of the unit first, second, and third drops that merged are also plotted. The probability of forming drop with a 6 ROs is approximately 6%, 9 ROs is approximately 13%, and 13 ROs is approximately 7%.

FIG. 3 depicts the results of the likelihood that a drop detected at a certain size is the result of a unit drop or merger given a particular mean for ROs loaded per unit drop. Each panel shows five peaks. Starting from the left, the first peak is a unit drop (an unmerged single droplet), the second peak is a droplet formed by merging two unit drops, the third peak is a droplet formed by merging three unit drops, the fourth peak is the result of merging five unit drops, and the fifth peak is the result of merging 10 unit drops. The first panel depicts probabilities for unit drops loaded with a mean of 1 RO per drop. The X axis is the number of ROs counted between 0 and 12 with increments of 2, the Y axis is the probability between 0% and 40% in increments of 10%, and the numbers on top indicate the size of the droplet detected relative to a unit drop between 0-10 and >10 in increments of one. The second panel depicts probabilities for unit drops loaded with a mean of 3 ROs per drop. The X axis is the number of ROs counted between 0 and 30 with increments of 10, the Y axis is the probability between 0% and 20% in increments of 10%, and the numbers on top indicate the size of the droplet detected relative to a unit drop between 0-10 and >10 in increments of one. The third panel depicts probabilities for unit drops loaded with a mean of 10 ROs per drop. The X axis is the number of ROs counted between 0 and 120 with increments of 20, the Y axis is the probability between 0% and 15% in increments of 5%, and the numbers on top indicate the size of the droplet detected relative to a unit drop between 0-10 and >10 in increments of one. The fourth panel depicts probabilities for unit drops loaded with a mean of 30 ROs per drop. The X axis is the number of ROs counted between 0 and 300 with increments of 100, the Y axis is the probability between 0% and 8% in increments of 2%, and the numbers on top indicate the size of the droplet detected relative to a unit drop between 0-10 and >10 in increments of one. The fifth panel depicts probabilities for unit drops loaded with a mean of 100 ROs per drop. The X axis is the number of ROs counted between 0 and 1200 with increments of 200, the Y axis is the probability between 0% and 4% in increments of 2%, and the numbers on top indicate the size of the droplet detected relative to a unit drop between 0-10 and >10 in increments of one. In each panel, the dotted lines between the sizes of the drop detected indicate the boundaries of the ranges in which a drop is called that size. For example, for the dotted line between 2 and 3, detecting any number of ROs to the left of the line will be considered as detecting a drop with 2× the volume of a unit drop (possibly the merger between two unit drops) and anything to the right of that line will be considered as detecting a droplet with 3× the volume of a unit drop (and possibly the merger of three unit drops). The figure shows that the ability to discriminate between the mergers of unit drops increases as the number of ROs per unit drop increases.

A “nucleic acid molecule” or “nucleic acid” as referred to herein refers to deoxyribonucleic acid (DNA) or ribonucleic acid (RNA) including known analogs or a combination thereof unless otherwise indicated. Nucleic acid molecules to be profiled herein are variously obtained from any source of nucleic acid. The nucleic acid molecule is alternately single-stranded or double-stranded. In some cases, the nucleic acid molecule is DNA. Categories of DNA contemplated herein include mitochondrial DNA, cell-free DNA, complementary DNA (cDNA), DNA circulating in an individual's bloodstream, environmental sample DNA, synthetic DNA or genomic DNA. Often, the nucleic acid is genomic DNA (gDNA), such as DNA isolated from a healthy or diseased tissue from an individual. In some cases the genomic DNA comprises at least one structural mutation, such as a translocation, duplication, deletion or insertion, or at least one point mutation such as a SNP, that is distinctive, correlative or causative of aberrant cell behavior such as cancer. Categories of DNA include plasmid DNA, cosmid DNA, bacterial artificial chromosomes (BAC), or yeast artificial chromosomes (YAC). The DNA variously is derived from at least one chromosome, up to a complete diploid or polyploid chromosome set. For example, if the DNA is from a human, the DNA is derived from at least one of chromosome 1, 2, 3, 4, 5, 6, 7, 8, 9, 10, 11, 12, 13, 14, 15, 16, 17, 18, 19, 20, 21, 22, X, and Y.

The RNA includes, but is not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs (miRNAs), siRNAs, piRNAs and long nc RNAs.

The nucleic acid molecules are often contained within at least one biological cell. Alternately, the nucleic acid molecules are contained within a noncellular biological entity, such as, for example, a virus or viral particle. Nucleic acid molecules are often constituents of a lysate of a biological cell or entity. Nucleic acid molecules are often profiled within a single biological cell or a single biological entity. Alternately, nucleic acid molecules are profiled in a lysate obtained from a single biological cell or a single biological entity. The source of nucleic acid for use in the methods and compositions described herein are often a sample comprising the nucleic acid.

The term “barcode” refers to a known nucleic acid sequence that allows some feature of a nucleic acid (e.g., oligo) with which the barcode is associated to be identified. A barcode sequence is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence is at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. An primer or adapter often comprises about, more than, less than, or at least 1, 2, 3, 4, 5, 6, 7, 8, 9, or 10 different barcodes. Barcodes can be of sufficient length and comprise sequences that are sufficiently different to allow the identification of biological molecules based on barcode(s) with which each biological molecule is associated.

As used herein, the term “about” a number refers to a range spanning that number plus or minus 10% of that number. The term “about” a range refers to that range minus 10% of its lowest value and plus 10% of its greatest value.

As used here, the terms “comprise,” “comprises,” “comprising,” “include,” “includes,” and “including” are interchangeable and not intended to be limiting, and refer to the nonexclusive presence of the recited element, leaving open the possibility that additional elements are present.

As used herein, the term “comparable to” a number refers to that number plus or minus 50% of that number. The term “comparable to” a range refers to that range minus 50% of its lowest value and plus 50% of its greatest value.

As used herein, “obtaining” a nucleic acid sample is given a broad meaning in some cases, such that it refers to receiving an isolated nucleic acid sample, as well as receiving a raw human or environmental sample, for example, and isolating nucleic acids therefrom.

“Solid supports” and “solid particles” are used interchangeably herein to refer to rigid or substantially rigid physical structures comprising one or more surfaces upon which one or more tags or labels can be positioned.

“Soft-gel beads” and “gel beads” are used interchangeably herein to refer to a bead comprising a solid support or particle encapsulated within a gel outer layer.

The terms “drop,” “droplet,” and “microdroplet” are used interchangeably herein and refer to discrete entities comprising an aqueous phase and one or more components encapsulated in the aqueous phase, and can have a longest dimension, such as a diameter, ranging from about 0.1 μm to about 1000 μm.

### EXAMPLES

The following examples are given for the purpose of illustrating various embodiments and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Evaluating Droplet Formation by Detecting Droplet Size or Merger Using Random Oligos and Barcodes

A sample is processed for barcoding using microfluidic methods and the droplets formed during the methods are evaluated for characteristics like size and merger. A pool of sample cells, barcode beads, and ROs are encapsulated at limiting dilution in droplets containing a lysis solution. Each RO comprises a highly randomized sequence flanked by a conserved sequence at each end. Each randomized sequence is synthesized by incorporating a mixture of all four nucleotides (A, T, G, C) and is generally about 10 bases in length. The conserved regions include either a forward or reverse primer binding site that allows for amplification of the oligo and the addition of a barcode.

The limiting dilution is such that droplets rarely receive more than a single cell or a single barcode bead. Each droplet also receives a distribution of ROs according to a Poisson distribution dependent upon the volume of the drop. The cells are not exposed to lysis solution until they are encapsulated in the droplets, ensuring that no lysis occurs prior to encapsulation. The lysis solution contains a non-ionic detergent and proteinase K. The droplets are incubated for up to 60 minutes at 55° C. and the cells are allowed to lyse and the proteinase K allowed to digest inhibitory proteins. The proteinase K is then inactivated by heating the droplets at 95° C. for 10 minutes.

The beads comprise ssDNA oligonucleotides containing a barcode and a primer configured to bind to the primer binding site on the random oligo. The barcodes are the same within each bead but different (while not necessarily unique) across beads. The concentration of primers is in excess of the amount necessary to label the sample and random oligonucleotides.

The drops are then merged with a second droplet containing standard reagents necessary for reverse transcription and PCR, including the necessary primers. A cDNA library is generated from the RNA in the cell. A forward and reverse primer binding site compatible with the subsequent barcoding step are added to each cDNA molecule during the reverse transcriptase step.

The ROs and cDNA are amplified by PCR. The ssDNA barcoded primers are bound to a bead via a photo-cleavable linker. The primers contain a first sequence that binds to the forward primer binding site on the ROs and the cDNA, a barcode, and a second sequence that provides a primer binding site compatible with standard Illumina sequencing. The resulting barcoded nucleotides are sequenced using next-generation sequencing approaches.

Sequencing reveals that each barcode bead labels a number of RO species consistent with the expected droplet volume. The barcodes within each droplet are identical, and thus each resulting amplicon within a droplet is labeled with the same, single barcode. Thus, it is concluded that the droplets did not merge, were approximately the desired volume, and that the cDNA sequences obtained with each barcode arose from the same droplet of origin.

### Example 2: Evaluating Droplet Formation by Detecting Droplet Size or Merger Using Random Oligos and Barcodes

A series of droplets are formed and processed as described in Example 1. Two droplets are sequenced, revealing that the first barcode bead labels three times as many RO species as the second barcode bead. The number of RO species labeled with the first barcode is also three times more than expected based on the target volume. Thus, it is concluded that the first droplet has three times the volume of the second droplet, as well as three times the target volume of each droplet. This increase in volume in the first droplet could be the result of a droplet being formed with three times the intended volume or the result of three droplets merging, only one of which contained a cell and only one of which contained a barcoded bead.

### Example 3: Evaluating Droplet Formation by Detecting Droplet Size or Merger Using Random Oligos and Barcodes

A series of droplets are formed and processed as described in Example 1. Two droplets are sequenced, revealing that the first barcode bead labels half as many RO species as the second barcode bead. The number of RO species labeled with the first barcode is also 50% less than expected based on the target volume. Thus, it is concluded that the first droplet has half the volume of the second droplet, as well as half the target volume of each droplet. This decrease in volume in the first droplet could be the result of a droplet being formed with half the intended volume.

### Example 4: Evaluating Droplet Formation by Detecting Droplet Size or Merger Using Random Oligos and Barcodes

A sample is processed for barcoding using microfluidic methods and the droplets formed during the methods are evaluated for characteristics like size and merger. A pool of sample cells and barcode beads are encapsulated in a set of first droplets at limiting dilution. The limiting dilution is such that most drops receive a single barcode bead and a single cell. The beads are the same as described in Example 1. The barcodes are the same within each bead and different, but not necessarily unique, across beads. The cells are lysed in the droplets as described in Example 1.

A set of PCR reagent droplets containing reagents necessary for cDNA generation and PCR amplification is formed. The PCR reagent droplets also contain a number of ROs consistent with the volume of the PCR reagent droplet according to a Poisson distribution. Each RO comprises a first portion comprising a conserved sequence and the second portion comprising a random sequence. The conserved regions allow for subsequent association with the barcode bead tag and PCR amplification. The random oligonucleotides are synthesized by incorporating a mixture of all four nucleotides (A, T, G, C) and are generally about 10 bases in length.

The first and second droplets are merged. A cDNA library is generated from the RNA in the cell as described in Example 1. The cDNA and ROs are amplified by PCR. Each resulting amplicon includes a droplet-specific barcode from the barcode from the barcode bead as described in Example 1.

After PCR amplification, two droplets are sequenced. Sequencing reveals that the ROs in each droplet are labeled in a droplet-specific manner. This result indicates that the droplets remained partitioned. Thus, it is concluded that the final droplets did not merge and the cDNA sequences labeled with the same barcodes arose from the same droplet.

Furthermore, the sequencing results reveal that each barcode bead labels a number of RO species consistent with the desired volume of the PCR reagent droplet. Thus, it is concluded that the PCR reagent droplets were approximately the desired volume.

### Example 5: Evaluating Droplet Formation by Detecting Droplet Size or Merger Using Random Oligos and Barcodes

Two droplets are each formed as described in Example 4. Sequencing reveals that amplicons of the same individual ROs can be found with different barcodes (i.e., a first amplicon of an RO is labeled with a first barcode and a second amplicon of the same RO is labeled with a second barcode). This result indicates that the template RO molecule was amplified in a first PCR cycle using a first barcode and in a second PCR cycle using a second barcode. As a result, it is concluded that two different barcode species were present in the same amplification droplet as a result of two barcode beads being present in the droplet. Additionally, the number of RO species labeled by each barcode is twice the expected number based on the target volume size. Thus, it is concluded that two droplets merged and the cDNA sequences obtained with each barcode are excluded from further analysis.

### Example 6: Evaluating Droplet Formation by Detecting Droplet Size or Merger Using Random Oligos and Barcodes

Two droplets are formed as described in Example 4. Sequencing reveals that amplicons of a first set of ROs are labeled with only a single droplet-specific barcode. Sequencing also reveals that a second set of ROs can be found with different barcodes (i.e., a first amplicon of an RO is labeled with a first barcode and a second amplicon of the same RO is labeled with a second barcode). The number of ROs found in each set is the same and roughly the same as the number that would be expected from droplets of the target droplet size. As a result, it is concluded that the first set of labeled ROs was generated in a first droplet with the target volume containing a single barcode bead. The first droplet remained partitioned during the amplification and labeling step. It is also concluded that the second droplet contained the appropriate final volume, but likely contained two different barcode beads or resulted from the merger of two smaller droplets. The cDNA sequences obtained from the first droplet are included in further analysis, while the cDNA sequences obtained from the second droplet are excluded.

### Example 7: Evaluating Droplet Formation by Detecting Droplet Size or Merger Using Random Oligos and Barcodes

Two droplets are each formed as described above in Example 4. Sequencing reveals that the sequences obtained from a first set of amplified ROs comprise the barcode from the first barcode bead and the sequences obtained from a second set of amplified ROs comprise the barcode from the second barcode bead. This result indicates that the RO template molecules in each droplet were amplified using the corresponding droplet-specific barcodes and, therefore, the droplets remained partitioned. Thus, it is concluded that the droplets did not merge and the cDNA sequences obtained with each barcode arose from the same droplet.

However, the sequencing results also reveal that the first barcode bead labels three times as many ROs as the second barcode bead. Furthermore, the number of RO species labeled with the first barcode is three times more than expected based on the target volume of each PCR reagent droplet according to a Poisson distribution. Thus, it is concluded that the volume of the PCR reagent droplet added to the droplet containing the first barcode bead was three times larger than intended, while the PCR reagent droplet added to the droplet containing the second barcode bead was the appropriate size.

### Example 8: Evaluating Droplet Formation and Tracking Droplet Workflow by Detecting Droplet Size or Merger Using Random Oligos and Barcodes

A sample is processed for barcoding using microfluidic methods and the droplets formed during the methods are evaluated for characteristics like size and merger. A pool of sample cells, barcode beads, and a first set of ROs are encapsulated in a set of first droplets at limiting dilution. Each RO in this first set comprises a first portion comprising a conserved sequence and the second portion comprising a random sequence. The conserved regions allow for subsequent association with the barcode bead tag and PCR amplification and also include an RO class-specific sequence that identifies these ROs as originating from the lysis droplet. This class-specific sequence is located between the primer binding site and the random oligo portion. The random oligonucleotides are synthesized by incorporating a mixture of all four nucleotides (A, T, G, C) and are generally about 10 bases in length.

The limiting dilution is such that most drops receive a single barcode bead and a single cell. The beads are the same as described in Example 1. The barcodes are the same within each bead and different, but not necessarily unique, across beads.

The cells are lysed in the droplets as described in Example 1.

A pool of PCR reagents necessary for cDNA generation and PCR amplification and a second set of ROs are encapsulated into a set of PCR reagent droplets. Each droplet is loaded with ROs according to a Poisson distribution based on the volume of the droplet. As with the first set of ROs, each RO in the second set comprises a first portion comprising a conserved sequence and the second portion comprising a random sequence. The conserved regions allow for subsequent association with the barcode bead tag and PCR amplification and also include an RO class-specific sequence that identifies these ROs as originating from the PCR reagent droplet. As a result, this class-specific sequence is different between the lysis droplet ROs and the PCR reagent droplet ROs. The random oligonucleotides are synthesized by incorporating a mixture of all four nucleotides (A, T, G, C) and are generally about 10 bases in length.

The first and second droplets are merged. A cDNA library is generated from the RNA in the cell as described in Example 1. The cDNA and ROs are amplified by PCR. Each resulting amplicon includes a droplet-specific barcode from the barcode from the barcode bead as described in Example 1.

After PCR amplification, two droplets are sequenced. Sequencing reveals that the ROs in each droplet are labeled in a droplet-specific manner. This result indicates that the droplets remained partitioned. Thus, it is concluded that the final droplets did not merge and the cDNA sequences labeled with the same barcodes arose from the same droplet.

Furthermore, the sequencing results reveal that each barcode bead labels a number of lysis RO species consistent with the desired volume of the lysis droplet and a number of PCR reagent droplet RO species consistent with the desired volume of the PCR reagent droplet. Thus, it is concluded that the droplets merged to form the final droplet were formed with approximately the desired volume.

### Example 9: Evaluating Droplet Formation and Tracking Droplet Workflow by Detecting Droplet Size or Merger Using Random Oligos and Barcodes

A series of droplets are formed as described in Example 8. A droplet is sequenced and it is determined that amplicons of individual RO species from both the lysis and PCR reagent droplet are labeled with two different barcodes. This result indicates that the template RO molecules were amplified during different PCR cycles with primers comprising different barcodes. As a result, it is concluded that two different barcode species were present in the same amplification droplet as a result of two barcode beads being present in the droplet.

Furthermore, the number of lysis droplet RO species and PCR reagent droplet RO species detected in the droplet are twice as high as expected based on the intended volume of each droplet. Thus, it is concluded that two amplification droplets, each containing a cell, a barcode bead, and PCR reagents and both lysis droplet ROs and PCR reagent droplet ROs merged prior to amplification. The cDNA sequences obtained with each barcode are excluded from further analysis.

### Example 10: Evaluating Droplet Formation and Tracking Droplet Workflow by Detecting Droplet Size or Merger Using Random Oligos and Barcodes

A series of droplets are formed as described in Example 8. A droplet is sequenced and it is determined that amplicons of individual RO species from both the lysis and PCR reagent droplet are labeled with the same barcode. This result indicates that there was only a single barcode bead present in the final amplification droplet.

The number of lysis droplet RO species detected in the droplet is the same as the number expected from a properly-formed lysis droplet with the appropriate volume. The number of RO species originating from the PCR reagent droplet, however, is three times higher than expected. Thus it is concluded that the final amplification droplet contained a single lysis droplet and three times the PCR reagent droplet volume as intended. This could have resulted from three PCR reagent droplets being merged with a single lysis droplet, three PCR reagent droplets merging with each other and the resulting droplet merging with the single lysis droplet, or a single PCR reagent droplet being formed with three times the intended volume being merged with a single lysis droplet.

### Example 11: Detecting Droplet Merger

A series of drops is evaluated. The drops were formed such that each droplet should receive an average of 10 random oligos, each of which contains the same conserved region. Each drop is shown in FIG. 1. R1, R2, etc. represent one set of random oligos with a common conserved region among R oligos. S1, S2, S3, etc. represent a different set of random oligos with a common conserved region among the S oligos, but different than the conserved region in the R oligos.

In FIG. 1A, a drop is counted with twelve ROs. It is inferred to be from a set of unit drops because twelve ROs is close to the average loading of ten.

In FIG. 1B, a drop is counted with 30 different RO species. Thus, it is inferred that this drop resulted from the merger of several unit drops—likely three drops.

In FIG. 1C, a drop is detected as containing 10 ROs from each unique set of ROs (R1, R2, R3 . . . and S1, S2, S3 . . . ). Thus, it is inferred to be the combination of two different drop types, each of a unit size.

In FIG. 1D, a drop is detected with three different sets of ROs (R, S, and T conserved regions). The drop contains 20 ROs with the R conserved region, 10 ROs with the S conserved region, and 10 ROs with the T conserved region. Thus, it is inferred that the drop resulted from the merger of four different drops. Two of those drops contained R ROs, one drop contained S ROs, and one drop contained T ROs.

### Example 12: Determining the Probability that a Droplet Resulted from a Merger

The impact of the RO loading rate λ, on drop identification is explored. In FIG. 2a, the mean occupancy of unit drops is three ROs. A drop with seven ROs is detected and the likelihood that it is a merger of three unit drops (inset, top row) or simply a statistically over packed, unmerged unit drop (inset below) is calculated using Formula I. The Poisson distributions of unit drops (λ=3) and 3-fold mergers (λ=9) are plotted. It is determined that about 12% of 3-fold mergers might have 7 ROs, whereas only 2% of unit drops will have that many. Thus, it is determined that the drop with seven ROs likely originated through a merger.

In FIG. 2b, the mean occupancy is 10 ROs per unit drop. It is determined that the resolving power improves dramatically for a drop containing a hypothetical 28 ROs, in part, because the peaks have narrowed. It is determined that a three-fold merger has a 7% chance of containing 28 ROs while a unit drop has effectively 0% chance. Thus, it is determined that the drop containing 28 ROs is very likely the result of a merger.

The calculations are repeated for various additional average numbers of random oligos loaded per drop, as shown in FIG. 3. A drop's size is determined based on its measured occupancy and the average unit occupancy. Ranges are set within which numbers of detected ROs are counted towards a certain size. This is depicted in FIG. 3a, which plots several distributions for possible occupancies given a drop of size m (i.e., the 1×, 2×, 3×, 5×, and 10× labeling each peak starting from the left, respectively) and unit occupancy A. For those distributions, a drop is called a size m (i.e., the size detected, as shown in the numbers at the top of each chart 1-10 and >10) if it falls in the range [(m−½)λ, (m+½)λ]. That range is demarcated with a dotted line.

The likelihood that a drop of some size is correctly called that size or some other is tabulated in FIG. 3b. For instance, given an average loading of 10 ROs per unit drop (λ=10), the measurement of a true unit drop will yield an empty drop 2.93% of the time, a unit drop 88.73% of the time, and a doublet 8.34% of the time. These numbers are outlined in the table. All of the drops are more easily resolved at an average loading of 100 ROs, a fact that is confirmed in the lower plot of FIG. 3a.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of evaluating a quality characteristic of a droplet comprising:
encapsulating a plurality of polynucleotides in a droplet, wherein the plurality of polynucleotides comprises at least one species of oligonucleotide;
tagging the polynucleotides with a label that identifies the polynucleotides as arising from the droplet;
counting a number of species of oligonucleotide tagged with the label; and determining a quality characteristic of the droplet based on the number of species of oligonucleotide tagged with the label.

2. The method of claim 1, wherein an oligonucleotide comprises:
a first nucleic acid segment and a second nucleic acid segment, wherein the first nucleic acid segment comprises a plurality of random nucleotides; and
the second nucleic acid segment comprises a conserved region common to the plurality of oligonucleotides.

3. The method of claim 2, further comprising forming the droplet in a microfluidic device.

4. The method of claim 2, wherein forming the droplet comprises isolating a portion of a first fluid, wherein the first fluid comprises a known concentration of oligonucleotide species.

5. The method of claim 4, wherein the first fluid comprises a plurality of the species of oligonucleotide dispersed throughout the first fluid, and wherein isolating the portion of the first fluid comprises isolating a number of oligonucleotide species in the droplet according to a Poisson distribution that is proportional to the volume of the droplet.

6. The method of claim 2, wherein the droplet comprises an aqueous phase fluid dispersed in an immiscible phase carrier fluid.

7. The method of claim 2, wherein the droplet comprises a known concentration of oligonucleotides.

8. The method of claim 2, wherein the plurality of polynucleotides comprises a number of polynucleotides encapsulated according to a Poisson distribution dependent on a volume of the droplet.

9. The method of claim 6, wherein the number of species of oligonucleotide tagged with the label is informative of a volume of the droplet.

10. The method of claim 9, wherein the volume of the droplet comprises a volume of the immiscible phase carrier fluid.

11. The method of any one of claims 1-10, wherein the plurality of polynucleotides further comprises polynucleotides obtained from a sample.

12. The method of claim 11, wherein the sample comprises a cell.

13. The method of claim 12, wherein the sample comprises no more than one cell.

14. The method of claim 11, wherein encapsulating the plurality of polynucleotides in the droplet comprises encapsulating a cell comprising polynucleotides in the droplet.

15. The method of claim 14, further comprising lysing the cell in the droplet.

16. The method of claim 2, wherein the label comprises a barcode.

17. The method of claim 14, wherein tagging the polynucleotides with a label comprises subjecting the droplet to conditions sufficient for enzymatic incorporation of the label into the plurality of polynucleotides.

18. The method of claim 17, wherein enzymatic incorporation of the label into the plurality of polynucleotides comprises ligating the label to the polynucleotides.

19. The method of claim 14, wherein tagging the polynucleotides with a label comprises subjecting the droplet to conditions sufficient for enzymatic incorporation of the label into amplification products of the plurality of polynucleotides.

20. The method of claim 19, wherein enzymatic incorporation of the label into amplification products of the plurality of polynucleotides comprises amplifying the plurality of polynucleotides by PCR using barcoded primers.

21. The method of any one of claim 2, wherein counting a number of species of oligonucleotide tagged with the label comprises sequencing the polynucleotides tagged with the label.

22. The method of claim 2, wherein data arising from the droplet is adjusted based on the quality characteristic.

23. The method of claim 22, wherein the quality characteristic comprises a volume of the droplet.

24. The method of claim 25, wherein the quality characteristic comprises a droplet merger.

25. The method of claim 2, wherein data arising from the droplet is excluded from further analysis based on the quality characteristic.

26. The method of claim 25, wherein the quality characteristic comprises a volume of the droplet.

27. The method of claim 25, wherein the quality characteristic comprises a droplet merger.

28. A method of evaluating a quality characteristic of a droplet comprising:
sequencing a plurality of polynucleotides obtained from the droplet, wherein the plurality of polynucleotides comprises at least one oligonucleotide species comprising a first nucleic acid segment and a second nucleic acid segment, wherein the first nucleic acid segment comprises a plurality of random nucleotides; and the second nucleic acid segment comprises a conserved region comprising a label,
detecting sequences of oligonucleotide species comprising labels; and determining a quality characteristic of the droplet based on the sequences of the oligonucleotide species detected.

29. The method of claim 28, wherein the plurality of polynucleotides comprises a first oligonucleotide species comprising a first conserved region and second oligonucleotide species comprising a second conserved region, wherein the detecting of sequences encoding the first oligonucleotide species and second oligonucleotide species is informative of a droplet merger.

30. The method of claim 29, wherein the first conserved region comprises a first label and the second conserved region comprises a second label.

31. The method of claim 30, wherein the first label comprises a first barcode and the second label comprises a second barcode.

32. The method of claim 29, wherein the first oligonucleotide species comprises a first label indicative of a first group of droplets and a droplet-specific label and the second oligonucleotide species comprises a second label indicative of a second group of droplets and the droplet specific label.
